(19) United States
(12) Patent Application Publication
(10) Pub. No.: US 2026/0036591 A1
(43) Pub. Date: Feb. 5, 2026

(54) METHODS FOR CHARACTERIZING COMPOSITIONS COMPRISING PEANUT ANTIGENS

(71) Applicant: SANOFI, Paris (FR)

(72) Inventors: Kim Alving, Paris (FR); Pierre deMontigny, Paris (FR); Bing Huang Wang, Paris (FR)

(21) Appl. No.: 19/268,299

(22) Filed: Jul. 14, 2025

Related U.S. Application Data
(62) Division of application No. 17/523,050, filed on Nov. 10, 2021, now Pat. No. 12,411,140.
Application No. 17/523,050 is a continuation of application No. 16/580,638, filed on Sep. 24, 2019, now Pat. No. 11,199,548.
Application No. 16/580,638 is a division of application No. 15/392,233, filed on Dec. 28, 2016, now Pat. No. 10,466,250.
(60) Provisional application No. 62/272,094, filed on Dec. 29, 2015.

(30) Foreign Application Priority Data
Dec. 8, 2016 (EP) 16306642.6

(51) Int. Cl. G01N 33/68

(52) U.S. Cl. CPC G01N 33/6848; G01N 2333/415

(57) ABSTRACT

Methods for determining an in vitro release profile of peanut allergens in a sample are provided. Methods for determining one or more signatures of peanut allergens in a sample are provided.

### RELATED APPLICATIONS

This application is a division of U.S. patent application Ser. No. 17/523,050, filed Nov. 10, 2021, which is a continuation of U.S. patent application Ser. No. 16/580,638, filed Sep. 24, 2019, now U.S. Pat. No. 11,199,548, which is a division of U.S. patent application Ser. No. 15/392,233, filed Dec. 28, 2016, now U.S. Pat. No. 10,466,250, which claims the benefit of U.S. Provisional Patent Application Ser. No. 62/272,094, filed Dec. 29, 2015, and European Patent Application No. 16306642.6, filed Dec. 8, 2016, each of which is incorporated herein by reference in its entirety.

### SEQUENCE LISTING

The instant application contains a Sequence Listing which has been submitted electronically in XML format and is hereby incorporated by reference in its entirety. Said XML file, created on Jul. 14, 2025, is named 767685_SA9-180DVCONDV_ST26.xml and is 228,412 bytes in size.

### FIELD OF THE INVENTION

The present invention relates to methods for characterizing e.g., determining release profiles, allergen signatures and the like, of therapeutic compositions comprising peanut allergens for use in treating, alleviating or otherwise reducing peanut allergy in a subject.

### BACKGROUND

Peanut allergy develops when the body's immune system has an abnormal hypersensitivity response to one or more peanut allergens. Peanut allergy is one of the most common food allergies in both children and adults. It receives particular attention because it is relatively common, typically lifelong, and can cause severe allergic reactions. Peanut allergy is the leading cause of anaphylaxis and death due to food allergy. It can lead to significant burden on patients and their families. Peanut is a common food ingredient making strict avoidance difficult. Therefore, there is a relatively high rate of accidental peanut ingestion for those trying to avoid peanuts. For these reasons, peanut allergy has become an important public health issue.

Research is currently underway focused on the development of compositions for the treatment of peanut allergy. Methods are needed for determining in vitro peanut allergen release data of known and newly developed compositions, both for quality control and to predict in vivo release profiles.

### SUMMARY OF THE INVENTION

The present invention is based in part on the discovery of highly sensitive methods to determine release profiles of allergens and/or to determine allergenic signatures of compositions comprising allergens. The methods described herein provide the sensitivity and accuracy needed to profile peanut allergens present in a sample, such as a protein extract, a therapeutic composition, or a dissolution or release medium and to allow measurement of low level, relevant allergens present in the sample. In exemplary embodiments, the methods include the step of detecting allergen digest products that exist in one or more isoforms of one or more allergens in the sample. The identification of allergen digest products found in multiple isoforms of a peanut allergen provides qualitative and quantitative information about a sample, which can be indicators of batch-to-batch consistency, and can provide a release profile of allergens stored on or in a substrate, such as, e.g., a nanoparticle.

In one aspect, the invention features a highly sensitive method for determining a signature of peanut allergens in a composition. The method includes the step of digesting peanut allergens present in a composition (e.g., medium or other sample) from the composition to generate allergen digest products, fragmenting the allergen digest products to generate peptide fragments, detecting and identifying the allergen digest products by mass spectrometry, and determining the signature of peanut allergens in the composition. In particular embodiments, the methods are used to determine a signature of one or more peanut allergens that are present in low concentrations in a composition. The signature includes the type and quantity (e.g., relative quantity) of allergens in the composition.

In certain embodiments, the composition is an aqueous medium, such as an aqueous pharmaceutical composition, an analytical sample, a dissolution medium or a release medium. In some embodiments, the total amount of peanut allergens in the composition is very low. For example, the amount of a particular peanut allergen (e.g., Ara h1, Ara h2,Ara h3 or Ara h6), may be less than about 2 μg/ml, less than about 1.5 μg/ml, less than about 1 μg/ml or less than about 0.5 μg/ml, e.g., about 0.2 μg/ml.

In some embodiments, the method further comprises the step of comparing a peanut allergen signature to a signature standard. The signature standard can be a peptide profile set by a regulatory authority, such as the FDA (Food and Drug Administration) or EMA (European Medicines Association), a peptide profile established by industry standards, or a profile set by expectations as determined by repeated experimentation. The signature standard can specify the types and relative amounts of peanut allergens that are expected to be found in a sample.

In certain exemplary embodiments, the allergen digest products are between about 4 amino acids and about 50 amino acids in length, between about 6 amino acids and about 30 amino acids in length, or between about 15 amino acids and about 20 amino acids in length, or are about 15, 16, 17, 18, 19 or 20 amino acids in length

In certain exemplary embodiments, the steps of fragmenting the allergen digest products and detecting the peptide fragments are performed by a method that includes tandem mass spectrometry, such as Liquid Chromatography-tandem Mass Spectroscopy (LC-MS-MS), nano tandem Mass Spectroscopy (nanoLC-MS-MS) or nano High Performance Liquid Chromatography-tandem Mass Spectroscopy (nanoHPLC-MS-MS).

In certain exemplary embodiments, the signature (e.g., peanut allergen signature and/or signature standard) comprises one or more Ara h1 allergen digest products having an amino acid sequence selected from the group consisting of SEQ ID NO:17, SEQ ID NO:70, SEQ ID NO:177, SEQ ID NO:155, SEQ ID NO:93 and SEQ ID NO:40. In some embodiments, the signature comprises allergen digest products from Ara h1, Ara h2 and Ara h6. In other embodiments, the signature comprises allergen digest products from Ara h1, Ara h2, Ara h3 and Ara h6. In some embodiments, the signature includes one or more allergen digest products having amino acid sequences selected from the group consisting of SEQ ID NO:17, SEQ ID NO:70, SEQ ID NO:177, SEQ ID NO:197, SEQ ID NO:198, SEQ ID NO:199, SEQ ID NO:200, SEQ ID NO:201, SEQ ID NO:236 and SEQ ID NO:237. In some embodiments, the signature includes one or more allergen digest products having amino acid sequences selected from the group consisting of SEQ ID NO:17, SEQ ID NO:70, SEQ ID NO:197, SEQ ID NO:198, SEQ ID NO:199, SEQ ID NO:200, SEQ ID NO:201, SEQ ID NO:236 and SEQ ID NO:237.

In some embodiments, allergen digest products suitable for use in establishing a peanut allergen profile are present in more than one isoform of a peanut allergen, such as in 2, 3, 4, 5, 6, 7, 8, 10, 12, 14, 16 or more isoforms of a particular allergen (e.g., an Ara h protein or polypeptide). For example, an allergen digest product suitable for use in a peanut allergen signature profile can be present in one or any combination of in 2, 3, 4, 5, 6, 7, 8 or more of isoforms of Ara h1, in 2, 3, 4, 5, 6, 7, 8 or more of isoforms of Ara h2, in 2, 3, 4, 5, 6, 7, 8 or more of isoforms of Ara h3, and/or in 2, 3, 4, 5, 6, 7, 8 or more of isoforms of Ara h6. In some embodiments, an allergen digest product suitable for use in a peanut allergen signature profile is present in all isoforms of one or more peanut allergens.

In certain exemplary embodiments, peanut allergens are digested with one or more proteases. For example, peanut allergens may be digested with one or more proteases selected from the group consisting of trypsin, endoproteinase Lys-C and endoproteinase Arg-C.

In certain exemplary embodiments, a composition comprising peanut allergens further comprises an internal standard. For example, in some embodiments, the internal standard comprises one or more heavy isotopes, such as ¹³C and/or ¹⁵N. In certain embodiments, the internal standard is not a full-length allergen, but is instead a fragment of the peptide. The fragment is typically less than 50 amino acids long (such as, e.g., between about 4 and about 50 amino acids long, between about 6 and about 30 amino acids long, or between about 10 and about 20 amino acids long). In some embodiments, the fragment has the sequence of an expected or predicted allergen digest product. In one embodiment, the internal standard is comprised of multiple peanut allergen peptides, each labeled at the C-terminus with a heavy isotope. For example, an exemplary internal standard can comprise any combination of two or more isotope-labeled fragments of an Ara h1 peptide, two or more isotope-labeled fragments of an Ara h2 peptide, two or more isotope-labeled fragments of an Ara h3 peptide and two or more isotope-labeled fragments of an Ara h6 peptide. In one embodiment, a composition comprising peanut antigens is digested with trypsin, a standard mix comprising ¹³C and/or ¹⁵N-labeled peanut allergen peptides is added to the digested mix, and then the composition is assayed by fragmentation, e.g., by mass spectrometry, such as e.g., LC-MS, or LC-MS-MS.

In certain exemplary embodiments, a signature comprises allergen digest products that do not contain missed proteolytic cleavage sites.

In another aspect, the invention features a method for determining a release profile, e.g., an in vitro release profile, of a composition comprising one or more peanut allergens. In one embodiment, the method includes obtaining one or more samples from the composition at each of a plurality of time points, digesting the peanut allergens present in the one or more samples to generate allergen digest products, fragmenting the allergen digest products to generate peptide fragments, and detecting the peptide fragments for at least two of the plurality of time points to determine the release profile of the peanut allergens. The composition can be, for example, a peanut extract, a therapeutic composition comprising peanut allergens, a dissolution medium or an analytical sample. In some embodiments the composition is an aqueous medium. In some embodiments, the amount of peanut allergens in the composition is very low. For example, the amount of a particular peanut allergen (e.g., Ara h1, Ara h2, Ara h3 or Ara h6), may be less than about 2 μg/ml, less than about 1.5 μg/ml, less than about 1 μg/ml or less than about 0.5 g/ml, e.g., about 0.2 μg/ml, or are about 15, 16, 17, 18, 19 or 20 amino acids in length. In some embodiments, allergens in a sample taken from a composition are digested to produce allergen digest products between about 4 amino acids and about 50 amino acids in length, between about 6 amino acids and about 30 amino acids in length, or between about 15 amino acids and about 20 amino acids in length. The pattern of allergen digest products obtained after digestion creates a peptide signature for the sample.

In some embodiments, the steps of fragmenting the allergen digest products and detecting the peptide fragments include one or more of a separation method and a peptide detection method. For example, in some embodiments, the steps of detecting and identifying the peptide fragments include performing methods such as LC-MS-MS, nano-LC-MS-MS, HPLC-MS-MS, or nanoHPLC-MS-MS. In some embodiments, the peptide signature includes a collection of fragments from one or more of peanut proteins Ara h1, Ara h2, Ara h3, Ara h4, Ara h5, Ara h6, Ara h7, Ara h8, Ara h9, Ara h10, Ara h11, Ara h12 and Ara h13.

In some embodiments, a peptide signature includes one or more Ara h1 allergen digest products having amino acid sequences selected from the group consisting of SEQ ID NO:17, SEQ ID NO:70, SEQ ID NO:177, SEQ ID NO:155, SEQ ID NO:93 and SEQ ID NO:40.

In some embodiments, the peptide signature comprises one or more allergen digest products from any combination of Ara h1, Ara h2 and Ara h6, or one or more allergen digest products from any combination of Ara h1, Ara h2, Ara h3 and Ara h6.

In some embodiments, the signature comprises one or more allergen digest products having amino acid sequences selected from the group consisting of SEQ ID NO:17, SEQ ID NO:70, SEQ ID NO:177, SEQ ID NO:197, SEQ ID NO:198, SEQ ID NO:199, SEQ ID NO:200, SEQ ID NO:201, SEQ ID NO:236 and SEQ ID NO:237.

In certain embodiments, allergen digest products are present in more than one isoform of a peanut allergen, such as in one, two, three, four or all isoforms of a peanut allergen, such as in one, two, three, four or all isoforms of Ara h1. In one embodiment, the allergen digest products are in more than one peanut allergen isoform.

In certain exemplary embodiments, the peanut allergens are digested with one or more proteases, such as one or more proteases selected from the group consisting of trypsin, endoproteinase Lys-C and endoproteinase Arg-C.

In certain embodiments, a sample, e.g., a sample taken from an extract or pharmaceutical composition, further comprises an internal standard. An internal standard is typically a peptide having the sequence of an expected or predicted allergen digest product, and is typically less than about 50 amino acids long (such as between about 4 and about 50 amino acids long, between about 6 and about 30 amino acids long, or between about 10 and about 20 amino acids long or about 10, about 11, about 12, about 13, about 14, about 15, about 16, about 17, about 18, about 19 or about 20 amino acids long). The internal standard can be added to the composition prior to sampling, and/or added to the sample prior to the enzymatic digestion step. In certain exemplary embodiments, the internal standard comprises one or more heavy isotopes, such as, e.g., ¹³C and/or ¹⁵N.

In some embodiments, the signature comprises allergen digest products that do not contain missed (uncleaved) proteolytic cleavage sites.

In some embodiments, the composition comprises a particle that encases the allergens and/or has allergens bound to the surface. The particle can be, for example, a nanoparticle, a microparticle, a film, a capsule or a hydrogel.

In some embodiments, the release profile is obtained over a period of time, such as, e.g., over a period of hours, e.g., a three-hour period of time, a six-hour period of time, a twelve-hour period of time, or a twenty-four hour period of time.

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1C depict a high quality Ara hl peptide (SEQ ID NO:70) identified by tryptic digestion followed by MS-MS. (A) depicts the sequence, the number of uncleaved trypsin cleavage sites (“missed”), the percentage of False Discovery Rate (FDR), the number of post-translational modification sites (“PTM”), confirmation in BLAST (Basic Local Alignment Tool) that the identified sequence is unique to the target protein (“BLAST”), the database searched, and the number of isoforms in the database that included the identified sequence. (B) and (C) depict MS-MS data in Table form and graphical form, respectively.

### DETAILED DESCRIPTION

The present invention is based in part on the development of sensitive analytical methods for measuring and profiling peanut allergens present in a composition, such as an extract or a therapeutic composition, and for determining a release profile, such as an in vitro release profile, from formulated compositions comprising peanut allergens. The methods include the detection of peptides that exist in one or more isoforms of one or more allergens in a sample. The identification of peptides found in multiple isoforms of a peanut allergen provides qualitative and quantitative information about a sample, which can be indicators of batch-to-batch consistency, and can provide a release profile of antigens stored on or in a substrate, such as a nanoparticle. The methods are particularly useful for profiling allergen content in compositions containing a very low amount of peanut allergens.

As used herein, the terms “peanut,” “groundnut” and “Arachis hypogea” are used interchangeably, and refer to a legume belonging to the Leguminosas family and the Papillionacea subfamily. Over 17 different peanut allergens have been identified. Peanut protein allergens include Ara h1, Ara h2, Ara h3, Ara H4, Ara h5, Ara h6, Ara h7, Ara h8, Ara h9, Ara h10, Ara h11, Ara h12, Ara h13, Ara h14, Ara h15, Ara h16 and Ara h17. GenBank Accession Numbers for the cDNA sequences of exemplary allergens include L34402.1 (Ara h1), AY007229.1 (Ara h2.0101), AY158467.1 (Ara h2.0201), AF093541.1 (Ara h3.0101), AF086821.1 (Ara h3.0201), AF059616 (Ara h5), AF092846.1 (Ara h6), AF091737.1 (Ara h7), EU046325.1 (AraH7.0201), AY328088.1 (Ara h8.0101), EF436550.1 (Ara h8.0201), EU159429.1 (Ara h9.0101), and EU161278.1 (Ara h9.0201), AY722694.2 (Ara h10.0101), AY722695.1 (Ara h10.0201), DQ097716.1 (Ara h11), EY396089.1 (Ara h12), EY396019.1 (AraH13), AAK13449 (Ara h14.0101), AAK13450 (Ara h14.0102), AAT11925 (Ara h14.0103), AAU21501 (Ara h15.0101), respectively (see, e.g., Leon et al., The peanut allergy epidemic: allergen molecular characterization and prospects for specific therapy. Expert Rev. Mol. Med. Vol. 9, Issue 1, January 2007; see also Arkwright et al., IgE Sensitization to the Nonspecific Lipid-Transfer Protein Ara h 9 and Peanut-Associated Bronchospasm, BioMed Research International, vol. 2013, Article ID 746507; see url address allergen.org/search.php?allergensource=Arachis+hypogaea).

In an exemplary embodiment, a signature of allergen digest products in a sample is obtained by digesting peanut allergens present in the sample to generate allergen digest products, fragmenting the allergen digest products to generate peptide fragments, and detecting and identifying the peptide fragments to obtain the signature of allergen digest products. As used herein, an “allergen digest product” refers to a peanut allergen present in an extract or sample that is digested, e.g., using an enzyme (e.g., trypsin, endoproteinase Lys-C, endoproteinase Arg-C and the like) to generate a product that is smaller than the intact allergen. The term “allergen digest product” also refers to the amino acid sequence of a peptide that would be produced if the peanut allergen were enzymatically digested.

As used herein, an “allergen” refers to a subset of antigens (e.g., peanut peptide antigens) which elicit the production of IgE in addition to other isotypes of antibodies. The terms “allergen,” “natural allergen,” and “wild-type allergen” may be used interchangeably.

As used herein, an “antigen” refers to a molecule (e.g., a peanut peptide) that elicits production of an antibody response (i.e., a humoral response) and/or an antigen-specific reaction with T-cells (i.e., a cellular response) in an animal.

Non-limiting examples of enzymes, specifically proteases, that are suitable to digest peanut allergens to generate allergen digest products include, but are not limited to, trypsin, endoproteinase Glu-C, endoproteinase Asp-N, chymotrypsin, endoproteinase Lys-C, and endoproteinase Arg-C, pepsin, papain, thermolysin, subtilisin, protease K, bromelain, sulfhydryl-specific protease (ficin) and the like.

As used herein, a “peptide fragment,” or “gas phase peptide fragment,” refers to any part or portion of the allergen that is smaller than the intact natural allergen that is generated by a fragmentation method that does not utilize an enzyme. Typically, fragmentation conditions are introduced in the gas phase, e.g., in a mass spectrometer step. In certain exemplary embodiments, a peptide fragment is less than 50 amino acids long, e.g., between about 2 and about 50 amino acids in length, between about 6 and about 30 amino acids in length, or between about 15 and about 20 amino acids in length or any values or sub-ranges within these ranges. In certain exemplary embodiments, a peptide fragment is about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, or about 49 amino acids in length.

In certain exemplary embodiments, a peptide fragment is generated by fragmenting an allergen digest product, and then using a separation process and a peptide identification method, such as Liquid Chromatography-tandem Mass Spectroscopy (LC-MS-MS), nano-LC-MS-MS, High Performance Liquid Chromatography-tandem MS (HPLC-MS-MS), nanoHPLC-MS-MS, UltraPerformance-tandem MS (UPLC-MS-MS), nanoUPLC-MS-MS, and Ultra High Performance-tandem MS (UHPLC-MS-MS), nanoUHPLC-MS-MS, or the like.

According to certain exemplary embodiments, a method described herein further comprises mass analyzing allergen digest products and/or peptide fragments using a mass analyzer. The mass analyzer typically comprises a triple quadrupole mass analyzer. According to other embodiments the mass analyzer may comprise a mass analyzer selected from the group consisting of: (i) triple quadrupole mass spectrometer, (ii) an orbitrap, such as a Fourier transform orbitrap, such as an Orbitrap ELITE™(Thermo Scientific); (iii) a Fourier Transform (“FT”) mass analyzer; (ii) a Fourier Transform Ion Cyclotron Resonance (“FTICR”) mass analyzer; (iii) a Time of Flight (“TOF”) mass analyzer; (iv) an orthogonal acceleration Time of Flight (“oaTOF”) mass analyzer; (v) an axial acceleration time of flight mass analyzer; (vi) a magnetic sector mass spectrometer; (vii) a Paul or 3D quadrupole mass analyzer; (viii) a 2D or linear quadrupole mass analyzer; (ix) a Penning trap mass analyzer; (x) an ion trap mass analyzer; and (xiii) an electrostatic Fourier transform mass spectrometer.

In certain exemplary embodiments, the methods described herein utilize a separation process such as a chromatography method, e.g., liquid chromatography. According to an embodiment, fragmenting the allergen digest products and/or detecting and identifying the peptide fragments is performed by: (i) High Performance Liquid Chromatography (“HPLC”), (ii) anion exchange, (iii) anion exchange chromatography; (iv) cation exchange; (v) cation exchange chromatography; (vi) ion pair reversed-phase chromatography; (vii) chromatography; (viii) single dimensional electrophoresis; (ix) multi-dimensional electrophoresis; (x) size exclusion; (xi) affinity; (xii) reverse phase chromatography; (xiii) Capillary Electrophoresis Chromatography (“CEC”); (xiv) electrophoresis; (xv) ion mobility separation; (xvi) Field Asymmetric Ion Mobility Separation or Spectrometry (“FAIMS”); (xvii) capillary electrophoresis; and (xviii) supercritical fluid chromatography.

According to certain exemplary embodiments, the method further comprises ionizing allergen digest products and/or peptide fragments in a sample to be analyzed. The ion source may comprise a continuous ion source. According to an embodiment, the ion source may be selected from the group consisting of: (i) an Electrospray ionization (“ESI”) ion source; (ii) a Matrix Assisted Laser Desorption Ionization (“MALDI”) ion source; (iii) a Desorption Ionization on Silicon (“DIOS”) ion source; and (iv) a Desorption Electrospray Ionization (“DESI”) ion source.

The peanut allergen signatures described herein are generally determined by measurement of multiple reaction monitoring (MRM) transitions consisting of the peptide precursor ion, one or more fragment ions and a retention time. This measurement is performed, for example, on a triple quadrupole instrument. The signature can also be obtained by a combination of retention time and accurate high resolution mass spectrometric analysis of the intact peptide. These quantitation methods typically require a labeled internal standard and an external synthetic peptide calibration curve. In one embodiment, a signature is obtained by a nano-LC/MS/MS (nLC-MS-MS) analysis where as many peptides as possible are fragmented and identified by database analysis and nLC/MS/MS datasets consisting of retention time and mass/charge values and intensity are measured and compared to determine global proteome changes.

In certain exemplary embodiments, internal standards are used that include a combination of two or more allergen digest products from protein allergens, such as from one or more of Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16 and Ara h17. The internal standards typically have a known peptide sequence and are provided in a known quantity. In some embodiments, the internal standards include a combination of allergen digest products from the peanut allergens Ara h1, Ara h2 and Ara h6. In other embodiments, internal standards are used that include a combination of allergen digest products from Ara h1, Ara h2, Ara h3 and Ara h6. In certain exemplary embodiments, a sample can include one or more internal standards that comprise one or any combination of allergen digest products listed in Tables 19-23. In some embodiments, the standards are labeled, such with one or more heavy isotopes, e.g., ¹³C or ¹⁵N.

In certain embodiments, the allergen digest products chosen to characterize the composition uniquely represent the peanut allergen or peanut allergen isoform family and are present in the majority or in all isoforms of the allergen it is derived from, enabling its usage for specific quantitation of the allergen in question, e.g., peanut allergens. Accordingly, in certain exemplary embodiments, a signature of allergen digest products is generated. As used herein, a “signature,” or “allergenic signature” refers to the presence of a particular combination and amount of specific peanut allergen digest products (e.g., Ara h1, Ara h2, Ara h6 and/or Ara h3 allergen digest products). In certain exemplary embodiments, a signature comprises 5, 6, 7, 8, 9, 10, 11, 12, 13, 14, 15, 16, 17, 18, 19, 20,21, 22, 23, 24, 25, 26, 27, 28, 29, 30, 35, 40, 45, 50, 55, 60, 65, 70, 75, 80, 85, 90, 95, 100 or more distinct allergen digest products, each distinct product having a unique sequence.

The selection of peptides for use in a peanut allergen signature can be based on the identification of isoforms that include the peptides identified by the steps of digestion and fragmentation. A variety of sequence databases can be used to identify the sequences of peanut allergen isoforms, including UniProt (online at uniprot.org, and which includes the Swiss-Prot and TrEMBL databases), the database at Allergen Nomenclature (online at allergen.org), GenBank (online at ncbi.nlm.nih.gov), GeneCards (online at genecards.org), Ensembl (online at ensemble.org), and the like.

Any sequence alignment algorithm can be used to identify the isoforms that include the peptides identified following digestion and fragmentation of a sample comprising peanut allergens. Exemplary sequence alignment algorithms include BLAST (online at blast.ncbi.nlm.nih.gov/Blast.cgi), Clustal Omega (online at ebi.ac.uk/Tools/msa/clustalo), and the like.

The peanut allergen profiling methods can be useful for a variety of purposes. For example, the methods described herein are useful for performing batch-to-batch reproducibility assessments in peanut-containing compositions. The methods described herein are also useful for measuring the release or leakage of peanut allergens from the surface or interior of a vessel or substrate, such as from a bead or a particle (e.g., a nanoparticle or microparticle), a capsule, a film or a strip (such as, e.g., for sublingual administration of the composition), or a gel (such as a hydrogel).

In some embodiments, the peptide signature includes peptides that include one or more immunogenic epitopes, or one or more immunodominant epitopes of one or more peanut allergens. Peptide signatures that comprise immunogenic epitopes or immunodominant epitopes can provide a standard for the immunogenicity of a composition comprising peanut allergens.

As used herein, a “sample” refers to any composition containing peanut allergens. Exemplary samples include, but are not limited to, peanut-containing extracts, peanut-containing powders, analytical samples comprising one or more peanut allergens, pharmaceutical compositions (e.g., therapeutic vaccines), dissolution or release media and the like. In typical embodiments, a sample will be aqueous.

A sample for use in the allergen profiling methods featured in the invention can be a peanut extract, such as an extract made from whole roasted or raw peanuts, or from peanut flour. The extract can be for use in a pharmaceutical composition, such as for the treatment or prevention of peanut allergy. The extract can be used in a composition for oral immunotherapy (OIT), or sublingual immunotherapy (SLIT), or in a composition for use in a nanoparticle composition, with or without an adjuvant. By assessing the peanut allergen signature of the peanut extract, batch-to-batch consistencies can be monitored. The peanut allergen signature can also be used as a factor to deduce the immunogenicity of the extract, as extracts with similar signatures are expected to have similar immunogenic properties.

In some embodiments, the amount of peanut allergens in the composition is very low. For example, the amount of a particular peanut allergen (e.g., Ara h1, Ara h2, Ara h3 and/or Ara h6), may be less than about 2 μg/ml, less than about 1.5 μg/ml, less than about 1 μg/ml or less than about 0.5 μg/ml, e.g., about 0.2 μg/ml.

Peanut protein extracts can be made by methods known in the art including defatting and/or filtration methods to produce peanut allergen preparations.

A sample for use in the allergen profiling methods described herein can be a therapeutic composition, such as a liquid formulation for administration orally, sublingually, mucosally, intradermally, subcutaneously, intravenously, intramuscularly, parenterally or by inhalation.

In some embodiments, a therapeutic composition will be in the form of a film or a strip. A sample can include a piece of the film dissolved in a buffer prior to analysis by the allergen profiling methods described herein.

In other embodiments, the sample will include a substrate, such as a nanoparticle, a capsule, a film or tablet, or a gel, such as a hydrogel. The allergen profiling methods described herein are useful to assay release of peanut allergens from the substrate, such as, e.g., from a nanoparticle or capsule, or from a film or a hydrogel. The release can be from the interior of the substrate, e.g., a particle, or from the exterior (e.g., a surface) of a substrate. In one embodiment, the release profile is assayed by performing a complete release of peanut allergens and then assaying for a controlled release, such as over a period of time or in different culture or solution conditions (e.g., at different temperatures, pH or the like). The amount of allergen released in the controlled release assay is typically reported as a fraction or percentage as compared to the amount of allergen released under the complete release conditions.

In certain exemplary embodiments, two or more signatures obtained at specific points in time can be used to determine an in vitro release profile of peanut allergens in a sample containing a substrate (e.g., an aqueous sample, e.g., a dissolution or release medium). An in vitro release profile can be determined over a period of hours, days or weeks. In certain exemplary embodiments, a release profile is obtained over a period of time of about 1, 2, 3, 4, 5, 6, 7, 8, 9, 10, 11, 12, 13, 14, 15, 16, 17, 18, 19, 20, 21, 22, 23 or about 24 hours. In certain exemplary embodiments, a release profile is obtained over a period of time of about 1, 2, 3, 4, 5, 6 or about 7 days. In certain exemplary embodiments, a release profile is obtained over a period of time of about one week, about two weeks, about three weeks, about four weeks, about five weeks, about six weeks, about seven weeks, or about eight weeks. In certain exemplary embodiments, 2, 3, 4, 5, 6, 7, 8, 9, 10, 11, 12, 13, 14, 15, 20, 25, 30, 35, 40, 45, 50 or more signatures are obtained over a particular period of time. For example, the release burst of peanut allergens can be assessed within the first 24 hours in evenly spaced time points with sufficient data to determine the burst kinetics followed by the release of the remaining amount of the same allergens from the sample to help assess overall product composition performance. The release profile can include types of allergens released from the substrate and/or the relative amounts of allergen released.

In one embodiment, the methods described herein indicate release of allergen from the surface of a substrate, e.g., a nanoparticle, by detecting a burst of peptides in a dissolution or release medium near the start of the assay. If allergen is present in the interior of the substrate, the detection of allergen over time will be more gradual according to the rate of release. Allergen that is both present on the surface of a substrate and encapsulated within the substrate may be identified by an initial burst of peptide detection in the dissolution or release medium near the start of the assay, as antigen from the surface is released from the substrate, followed by a more gradual increase in peptide detection in the dissolution or release media as allergen is released (e.g., leaked) from the interior of the substrate.

As used herein, “dissolution medium,” “dissolution media,” “release medium” and “release media” refer to a composition that is used to provide in vitro drug release information. Dissolution or release media is useful, for example, for quality control testing of a sample for determining the release and/or stability of allergen in the sample. In choosing a suitable dissolution or release medium, it is useful to determine the analytical target profile of the allergen (e.g., delayed release, constant release, extended release and the like) and/or the allergen solubility profile. For a review of dissolution media selection, see Martin and Gray (Summer 2011) Journal of Validation Technology.

As used herein, “release rate” refers to the rate that an entrapped peanut allergen agent flows from a composition and into a surrounding medium in an in vitro release test. In one exemplary embodiment, the composition is first prepared for release testing by placing the composition into the appropriate in vitro release medium. This is generally performed by exchanging the buffer after centrifugation to pellet the substrate (e.g., a synthetic nanocarrier), and reconstituting of the substrate using mild conditions. In certain embodiments, the assay is started by placing the sample at 37° C. in an appropriate temperature-controlled apparatus. A sample is typically removed at various time points.

As used herein, a “release profile” refers to the types of proteins and/or peptides, e.g., peanut allergens, that are released from a substrate or vessel over time, and may also include the rate at which each particular protein, peptide and/or allergen in or on the substrate or vessel is released.

“Exhibits a pH-sensitive dissociation” refers to a coupling between two entities, such as a peanut allergen and a substrate, e.g., a carrier molecule, that is significantly reduced or eliminated by exposure of the two entities to a change in environmental pH. In certain embodiments, relevant pH-sensitive dissociations may satisfy any of the relationships or combinations thereof provided herein.

In certain exemplary embodiments, a pharmaceutical composition comprises nanocarriers and/or microcarriers, such as synthetic nanocarriers and/or synthetic microcarriers. As used herein, a “synthetic nanocarrier” or “synthetic microcarrier” refers to a discrete object that possesses at least one dimension that is less than or equal to 5 microns in size.

In some embodiments, the mass balance is compared between one or more peanut allergens in one or more compositions to be compared, such as in a sampling from a composition over a period of time across different time points, or between different batches made at different times, and/or by different methods. The comparative data can be presented as a relative quantitation, and are typically expressed as a fraction or as a percentage.

Pharmaceutical compositions containing peanut allergens can generally be formulated with carriers, excipients, and other agents that provide suitable transfer, delivery, tolerance, and the like. Exemplary formulations include, for example, powders, pastes, ointments, jellies, waxes, oils, lipids, lipid (cationic or anionic) containing vesicles (such as LIPOFECTIN™), anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J. Pharm Sci. Technol. 52:238-311.

“Pharmaceutically acceptable excipient” refers to a pharmacologically inactive substance added to a composition (e.g., a therapeutic composition) to further facilitate administration of the composition. Pharmaceutically acceptable excipients include, but are not limited to, calcium carbonate, calcium phosphate, various diluents, various sugars and types of starch, cellulose derivatives, gelatin, vegetable oils, polyethylene glycols and the like.

“Dosage form” refers to a drug (e.g., one or more peanut allergens) in a medium, carrier, vehicle or device suitable for administration to a subject.

“Effective amount” refers to that amount effective for a certain purpose. For example, when the effective amount is for a therapeutic purpose, an effective amount is an amount that treats, alleviates, ameliorates, relieves, delays onset of, inhibits progression of, reduces severity of and/or reduces incidence of one or more symptoms or features of a disease, disorder and/or condition provided herein, e.g., a peanut allergy.

“Subject” refers an animal, including mammals such as humans and non-human primates; avians; domestic household or farm animals such as cats, dogs, sheep, goats, cattle, horses and pigs; laboratory animals such as mice, rats and guinea pigs; fish; and the like.

As used herein, “vaccine” refers to a composition of matter that improves the immune response to a particular disease or disorder. A vaccine typically contains factors that stimulate a subject's immune system to recognize a specific antigen (e.g., a peanut antigen) as foreign and eliminate it from the subject's body. A vaccine also establishes an immunologic “memory” such the antigen will be quickly recognized and responded to if a subject is re-challenged. Vaccines can be prophylactic (for example to prevent future infection by any pathogen) or therapeutic (for example a vaccine against a peanut antigen for the treatment of peanut allergy).

“Administering” or “administration” means providing a drug to a subject in a manner that is pharmacologically useful.

As used herein, an “epitope” refers to a binding site including an amino acid motif of between approximately six and fifteen amino acids which can be bound by an immunoglobulin (e.g., IgE, IgG, etc.) or recognized by a T-cell receptor when presented by an APC in conjunction with the major histocompatibility complex (MHC). A linear epitope is one where the amino acids are recognized in the context of a simple linear sequence. A conformational epitope is one where the amino acids are recognized in the context of a particular three dimensional structure. The peanut allergen signatures identified by the methods featured in the invention may comprise one or more peanut epitopes. An immunogenic epitope can provoke an immune response in the body, e.g., an allergic response.

As used herein, an “immunodominant epitope” refers to an epitope which is bound by antibody in a large percentage of the sensitized population or where the titer of the antibody is high, relative to the percentage or titer of antibody reaction to other epitopes present in the same antigen. In one embodiment, an immunodominant epitope is bound by antibody in more than 50% of the sensitive population, more preferably more than 60%, 70%, 80%, 90%, 95%, or 99% of the sensitive population. The peanut allergen signatures identified by the methods featured in the invention will typically comprising one or more peanut immunodominant epitopes.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following example, which is included for purposes of illustration only and is not intended to be limiting.

### EXAMPLE I

### Peanut Allergen Signature

The development and initial preliminary validation of a method for the relative quantitation of four peanut allergens, Ara h1, Ara h2, Ara h3 and Ara h6, are described below. The method utilized liquid chromatography coupled to tandem mass spectrometry, and was based on the measurement of representative tryptic peptides derived from each of these proteins. The peptides were chosen in each case to include the majority of the reported allergenic protein isoforms.

The profiling method was dependent on the digest reproducibility and digest efficiency for the protein of interest in a complex protein mixture. Not all proteolytic peptides are good candidates for quantitation, and whereas good ionization efficiency and consistent tryptic cleavage, as well as low probability of the peptide harboring post-translational modification, are important aspects of the selection process, issues such as interference from other peptides and other matrix effects in a complex digest, are more difficult to predict. Once selected, a series of both heavy isotope labeled peptides and non-labeled synthetic versions of the selected peptides were procured and used as internal standards and for buffer calibration curves, respectively.

Peanut extracts prepared from lightly roasted peanut flour (Golden Peanut and Tree Nuts, Alpharetta, GA) were digested in quadruplicate with trypsin, and then a standard was added to the digested sample. The standard contained two Ara hl peptides, two Ara h2 peptides, two Ara h3 peptides, and two Ara h6 peptides. The peptides in the internal standard were labeled with one or both of ¹³C and ¹⁵N. The sequences of the internal standards are provided in Tables 1 and 2 below.

TABLE 1
Unlabeled synthetic “Light” peptides for
internal standard

| Peptide sequence | peptide | formula |
|---|---|---|
| DLAFPGSGEQVEK | ARA_L1-1 | C60 H93 N15 O22 |
| (SEQ ID NO: 238) | | |
| GTGNLELVAVR | ARA_L1-2 | C48 H85 N15 O16 |
| (SEQ ID NO: 239) | | |
| GAGSSQHQER | ARA_L2-1 | C40 H65 N17 O17 |
| (SEQ ID NO: 240) | | |
| QQEQQFK | ARA_L2-2 | C40 H62 N12 O14 |
| (SEQ ID NO: 241) | | |
| RPFYSNAPQEIFIQQGR | ARA_L3-1 | C93 H139 N27 O26 |
| (SEQ ID NO: 242) | | |
| AHVQVVDSNGNR | ARA_L3-2 | C52 H86 N20 O19 |
| (SEQ ID NO: 243) | | |
| IMGEQEQYDSYDIR | ARA_L6-1 | C74 H111 N19 O28 S1 |
| (SEQ ID NO: 244) | | |
| QMVQQFK | ARA_L6-2 | C40 H65 N11 O11 S1 |
| (SEQ ID NO: 245) | | |

TABLE 2
Labeled synthetic “Heavy” peptides for
internal standard

| Peptide sequence | peptide | formula |
|---|---|---|
| DLAFPGSGEQVEK* | ARA_H1-1 | 13C6 C54 H93 15N2 |
| (SEQ ID NO: 246) | | N13 O22 |
| GTGNLELVAVR* | ARA_H1-2 | 13C6 C42 H85 15N4 |
| (SEQ ID NO: 247) | | N11 O16 |
| GAGSSQHQER* | ARA_H2-1 | 13C6 C34 H65 15N4 |
| (SEQ ID NO: 248) | | N13 O17 |
| QQEQQFK* | ARA_H2-2 | 13C6 C34 H62 15N2 |
| (SEQ ID NO: 249) | | N10 O14 |
| RPFYSNAPQEIFIQQGR* | ARA_H3-1 | 13C6 C87 H139 15N4 |
| (SEQ ID NO: 250) | | N23 O26 |
| AHVQVVDSNGNR* | ARA_H3-2 | 13C6 C46 H86 15N4 |
| (SEQ ID NO: 251) | | N16 O19 |
| IMGEQEQYDSYDIR* | ARA_H6-1 | 13C6 C68 H111 15N4 |
| (SEQ ID NO: 252) | | N14 O28 S1 |
| QMVQQFK* | ARA_H6-2 | 13C6 C34 H65 15N4 |
| (SEQ ID NO: 253) | | N7 O11 S1 |

K* and R* are heavy isotope labeled amino acids where the carbon-12 has been replaced with carbon-13 and nitrogen-14 with nitrogen-15 resulting in 13C6 15N2 for lysine and 13C6 15N4 for arginine

Notably, additional peptides could have been selected and the particular characteristics of the proteins made it difficult to fulfill all desirable features for all peptides. For example, the two peptides for Ara h6 contain methionine, which is undesirable but difficult to avoid as Ara h6 contains an unusually high content of S-containing amino acids. Similarly, Ara h3 contains N-terminal “missed” cleavage sites that have been found to be stable. Often, tryptic cleavage sites that are very close to each other will result in preferential cleavage of one of the sites, not both.

The internal standards were used to quantitate the digested products. Buffer calibration curves of synthetic unlabeled signature peptides were generated with heavy isotope-labeled internal standards added to both the peanut extract digest and the calibration curve. Relative quantification was performed applying the internal standard and calibration curve to determine the relative amount of signature peptides in the peanut extract digest.

The samples containing the digested test allergens and the standards were analyzed by nanoHPLC-MS-MS gas phase fragmentation (Orbitrap ELITE™, ThermoScientific). Using the top-15 method, one sample (out of the four total) was analyzed three times, and the remaining three samples were analyzed one time, to generate a total of six data files. These data files were analyzed by Proteome Discoverer™ Software (Thermo Scientific). Applying the top-15 method to identify the most abundant proteins assured that peptides of high ionization and fragmentation efficiency would be identified in a consistent manner across the data sets.

The identities of the resulting fragments were determined using the UniProt sequence database at uniprot.org.

Over 90% of all known peanut allergens were detected from the peanut flour sample, including Ara h1, Ara h2, Ara h3, Ara h5, Ara h6, Ara h7, Ara h8, Ara h10, Ara h11, Ara h14, Ara h15, and Ara h Agglutinin. A survey of the identified allergens is illustrated in Tables 3-18 below.

Ara h1, Ara h2, Ara h3 and Ara h6 represent the major peanut allergens.

TABLE 3
Ara h1 isoforms detected in peanut flour.

| | | Peak Identified by |
|---|---|---|
| Peanut Allergen | UniProt Ref | nanoHPLC-MS-MS |
| Ara h 1 | B3IXL2 | No |
| Ara h 1 | E5G076 | Yes |
| Ara h 1 | N1NEW2 | No |
| Ara h 1 | N1NG13 | No |
| Ara h 1 | P43237 | Yes |
| Ara h 1 | Q6PSU3 | Yes |
| Ara h 1 | Q6PSU4 | Yes |
| Ara h 1 | Q6PSU5 | Yes |
| Ara h 1 | Q6PSU6 | Yes |
| Ara h 1.0101 | P43238 | Yes |

TABLE 4
Ara h2 isoforms detected in peanut flour.

| | | Peak Identified by |
|---|---|---|
| Peanut Allergen | UniProt Ref | nanoHPLC-MS-MS |
| Ara h 2 | C0LJJ1 | No |
| Ara h 2.0101 | Q6PSU2 | Yes |
| Ara h 2.0201 | Q6PSU2 | Yes |

TABLE 5
Ara h3 isoforms detected in peanut flour

| | | Peak Identified by |
|---|---|---|
| Peanut Allergen | UniProt Ref | nanoHPLC-MS-MS |
| Ara h 3 | A1DZF0 | Yes |
| Ara h 3 | A1DZF1 | Yes |
| Ara h 3 | B5TYU1 | Yes |
| Ara h 3 | E5G077 | Yes |
| Ara h 3 | Q0GM57 | Yes |
| Ara h 3 | Q5I6T2 | Yes |
| Ara h 3 | Q647H2 | Yes |
| Ara h 3 | Q647H3 | Yes |
| Ara h 3 | Q647H4 | Yes |
| Ara h 3 | Q6IWG5 | Yes |
| Ara h 3 | Q6T2T4 | Yes |
| Ara h 3 | Q8LKN1 | Yes |
| Ara h 3 | Q8LL03 | Yes |
| Ara h 3 | Q9FZ11 | Yes |
| Ara h 3.0101 | O82580 | Yes |
| Ara h 3.0201 | Q9SQH7 | Yes |
| n/a | O82580 | Yes |
| n/a | Q9SQH7 | Yes |

TABLE 6
Ara h5 isoforms identified in peanut flour

| | | Peak Identified by |
|---|---|---|
| Peanut Allergen | UniProt Ref | nanoHPLC-MS-MS |
| Ara h 5 | D3K177 | Yes |
| Ara h 5 | L7QH52 | Yes |
| Ara h 5 | Q5XXQ5 | Yes |
| Ara h 5.0101 | Q9SQI9 | Yes |

TABLE 7
Ara h6 isoforms identified in peanut flour

| | | Peak Identified by |
|---|---|---|
| Peanut Allergen | UniProt Ref | nanoHPLC-MS-MS |
| Ara h 6 | A1DZE9 | Yes |
| Ara h 6 | A5Z1R0 | No |
| Ara h 6.0101 | Q647G9 | Yes |

TABLE 8
Ara h7 isoforms identified in peanut flour

| | | Peak Identified by |
|---|---|---|
| Peanut Allergen | UniProt Ref | nanoHPLC-MS-MS |
| Ara h 7 | Q647G8 | Yes |
| Ara h 7.0101 | Q9SQH1 | Yes |
| Ara h 7.0201 | B4XID4 | Yes |

TABLE 9
Ara h8 isoforms identified in peanut flour

| | | Peak Identified by |
|---|---|---|
| Peanut Allergen | UniProt Ref | nanoHPLC-MS-MS |
| Ara h 8 | B1PYZ4 | Yes |
| Ara h 8 | B2ZGS2 | Yes |
| Ara h 8 | Q0PKR4 | Yes |
| Ara h 8 | Q2YHR1 | Yes |
| Ara h 8.0101 | Q6VT83 | Yes |
| Ara h 8.0201 | B0YIU5 | Yes |

TABLE 10
Ara h9 isoforms identified in peanut flour

| | | Peak Identified by |
|---|---|---|
| Peanut Allergen | UniProt Ref | nanoHPLC-MS-MS |
| Ara h 9.0101 | B6CEX8 | Yes |
| Ara h 9.0201 | B6CG41 | Yes |

TABLE 11
Ara h10 isoforms identified in peanut flour.

| | | Peak Identified by |
|---|---|---|
| Peanut Allergen | UniProt Ref | nanoHPLC-MS-MS |
| Ara h 10.0101 | Q647G5 | Yes |
| Ara h 10.0102 | Q647G4 | Yes |

TABLE 12
Ara h11 isoforms identified in peanut flour.

| | | Peak Identified by |
|---|---|---|
| Peanut Allergen | UniProt Ref | nanoHPLC-MS-MS |
| Ara h 11.0101 | Q45W87 | Yes |
| Ara h 11.0102 | Q45W86 | Yes |

TABLE 13
Ara h12 isoforms identified in peanut flour.

| | | Peak Identified by |
|---|---|---|
| Peanut Allergen | UniProt Ref | nanoHPLC-MS-MS |
| Ara h 12.0101 | EY396089 | No |

TABLE 14
Ara h13 isoforms identified in peanut flour.

| | | Peak Identified by |
|---|---|---|
| Peanut Allergen | UniProt Ref | nanoHPLC-MS-MS |
| Ara h 13.0101 | EY396019 | No |

TABLE 15
Ara h14 isoforms identified in peanut flour.

| | | Peak Identified by |
|---|---|---|
| Peanut Allergen | UniProt Ref | nanoHPLC-MS-MS |
| Ara h 14.0101 | Q9AXI1 | Yes |
| Ara h 14.0102 | Q9AXI0 | Yes |
| Ara h 14.0103 | Q6J1J8 | Yes |

TABLE 16
Ara h15 isoforms identified in peanut flour.

| | | Peak Identified by |
|---|---|---|
| Peanut Allergen | UniProt Ref | nanoHPLC-MS-MS |
| Ara h 15.0101 | Q647G3 | Yes |

TABLE 17
Ara h Agglutinin isoforms identified in peanut flour.

| | | Peak Identified by |
|---|---|---|
| Peanut Allergen | UniProt Ref | nanoHPLC-MS-MS |
| Ara h Agglutinin | P02872 | Yes |
| Ara h Agglutinin | Q38711 | Yes |
| Ara h Agglutinin | Q43373 | Yes |
| Ara h Agglutinin | Q43375 | Yes |
| Ara h Agglutinin | Q8W0P8 | Yes |

TABLE 18
Ara h hypothetical isoforms identified in peanut flour.

| Hypothetical | | Peak Identified by |
|---|---|---|
| Peanut Allergen | UniProt Ref | nanoHPLC-MS-MS |
| Ara i 2 | A5Z1Q9 | No |
| Ara i 6 | A5Z1Q6 | No |
| Ara d 2 | A5Z1Q8 | No |
| Ara d 2 | A8VT41 | No |
| Ara d 2 | A8VT44 | No |
| Ara d 2 | A8VT45 | No |
| Ara d 2 | A8VT50 | No |
| Ara d 6 | A5Z1Q5 | No |

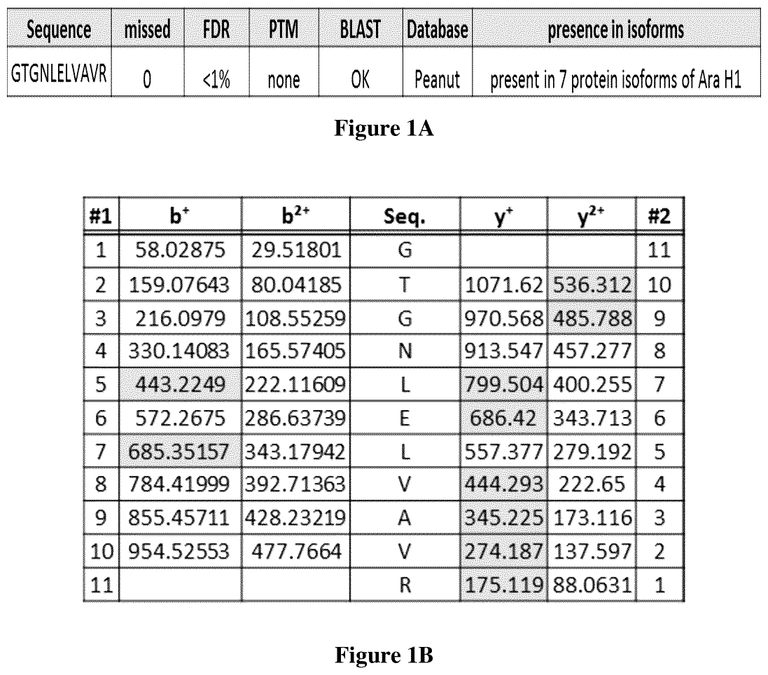

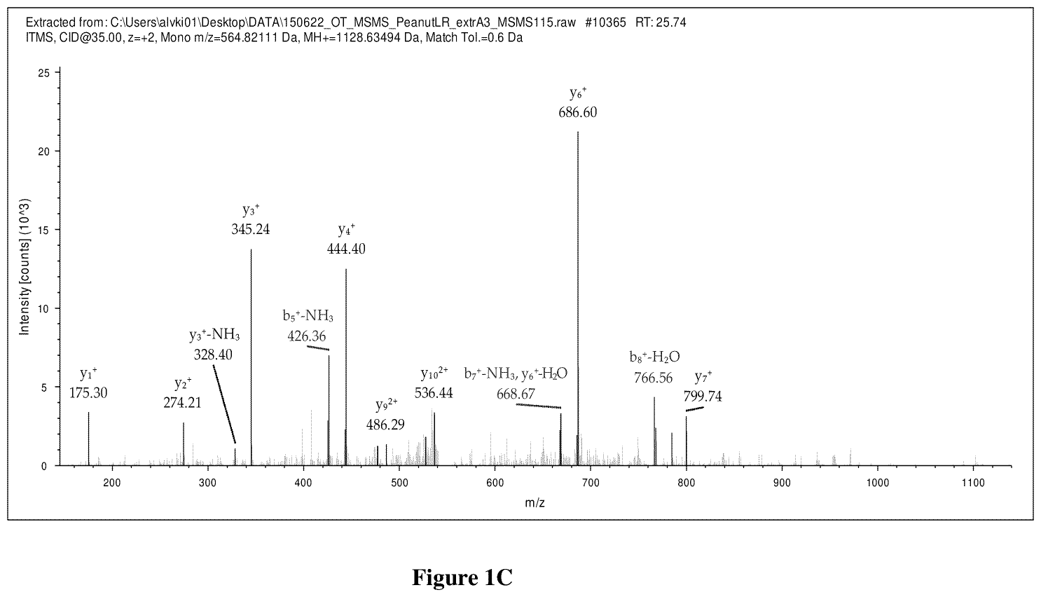

Peptides of the major allergens are identified to form a collection of peptides for use as an indicator of peanut allergen content in a composition (e.g., a “peanut peptide signature”). An ideal peptide signature utilizes peptides that represent completely cleaved true tryptic digest products (with the possible exception of peptides that contain sequential arginine and/or lysine residues), where one or the other cleavage site is typically preferentially cleaved. Selected peptides also typically have sequence conservation with a False Discovery Rate (FDR) of less than 1%, and sequence conservation across multiple isoforms. Further, selected peptides typically have no or minimal post-translational modifications, including oxidation and glycosylation, and have high detection quality based on manual curation of tandem mass spectrometry (MS-MS). Isoforms are represented by genetic variations, including peptides generated by truncated or deleted sequences. An example of a high quality peptide as identified by MS-MS is the Ara h1 peptide illustrated in FIGS. 1A to 1C.

For example, Ara h1k heterogeneity was determined by comparing sequence variations in the UniProt database records. Exemplary Ara h1 allergen digest products identified from UniProt reference P43238 are shown in Table 19. Sequences from other UniProt Ara h1 records were also compared (see Table 20). Preferred Ara h1 allergen digest products were selected as being useful in a peanut allergen signature based on: 1) preferentially zero missed tryptic cleavage sites; 2) presence in all sequences with a False Discovery Rate (FDR) of less than 1%; 3) no or minimal post-translational modification sites; 4) high quality fragments as determined by manual curation of tandem Mass Spectroscopy (MS-MS) data; 5) an indication by BLAST search that the sequence is unique within the peanut proteome; and 6) presence of the sequence a maximum number of isoforms. The resulting initial list of allergen digest products that were determined to be most useful candidates for profiling peanut antigens in the peanut flour extract are shown in Tables 20-23, representing peanut allergens from Ara h1, Ara h2, Ara h3 and Ara h6, respectively.

TABLE 19
Ara h1 allergen digest products from UniProt
Ref. P43238.

| | SEQ |
|---|---|
| | ID |
| Ara h1 Peptide Sequence | NO: |
| ACESRCTKLEYDPR | 1 |
| ACESRCTKLEYDPRCVYDPR | 2 |
| AMVIVVVNK | 3 |
| AMVIVVVNKGTGNLELVAVR | 4 |
| AMVIVVVNKGTGNLELVAVRK | 5 |
| CLQSCQQEPDDLK | 6 |
| CLQSCQQEPDDLKQK | 7 |
| CLQSCQQEPDDLKQKACESR | 8 |
| CLQSCQQEPDDLKQKACESRCTK | 9 |
| CTKLEYDPR | 10 |
| CTKLEYDPRCVYDPR | 11 |
| CTKLEYDPRCVYDPRGHTGTTNQR | 12 |
| CVYDPR | 13 |
| CVYDPRGHTGTTNQR | 14 |
| CVYDPRGHTGTTNQRSPPGER | 15 |
| CVYDPRGHTGTTNQRSPPGERTR | 16 |
| DLAFPGSGEQVEK | 17 |
| DLAFPGSGEQVEKLIK | 18 |
| DNVIDQIEKQAK | 19 |
| DQSSYLQGFSR | 20 |
| DQSSYLQGFSRNTLEAAFNAEFNEIRR | 21 |
| EDQEEENQGGKGPLLSILK | 22 |
| EDWRRPSHQQPR | 23 |
| EEDWRQPREDWR | 24 |
| EEDWRQPREDWRRPSHQQPR | 25 |
| EEEEDEDEEEEGSNREVRRYTAR | 26 |
| EEGGRWGPAGPR | 27 |
| EEGGRWGPAGPRER | 28 |
| EEGGRWGPAGPRERER | 29 |
| EETSRNNPFYFPSR | 30 |
| EETSRNNPFYFPSRR | 31 |
| EETSRNNPFYFPSRRFSTR | 32 |
| EGALMLPHFNSK | 33 |
| EGALMLPHFNSKAMVIVVVNK | 34 |
| EGDVFIMPAAHPVAINASSELHLLGFGINAENNHRIFLAGDK | 35 |
| EGDVFIMPAAHPVAINASSELHLLGFGINAENNHRIFLAGDK | 36 |
| DNVIDQIEK | |
| EGEPDLSNNFGK | 37 |
| EGEPDLSNNFGKLFEVKPDKK | 38 |
| EGEPDLSNNFGKLFEVKPDKKNPQLQDLDMMLTCVEIK | 39 |
| EGEQEWGTPGSHVR | 40 |
| EGEQEWGTPGSHVREETSRNNPFYFPSRR | 41 |
| EHVEELTK | 42 |
| EHVEELTKHAK | 43 |
| EQQQRGRR | 44 |
| EQQQRGRREEEEDEDEEEEGSNR | 45 |
| EREEDWR | 46 |
| EREEDWRQPR | 47 |
| EREREEDWR | 48 |
| EREREEDWRQPR | 49 |
| ESHFVSARPQSQSQSPSSPEK | 50 |
| ESHFVSARPQSQSQSPSSPEKESPEKEDQEEENQGGK | 51 |
| ESPEKEDQEEENQGGK | 52 |
| EVRRYTAR | 53 |
| FDQRSR | 54 |
| FDQRSRQFQNLQNHR | 55 |
| FSTRYGNQNGR | 56 |
| FSTRYGNQNGRIR | 57 |
| FSTRYGNQNGRIR | 58 |
| GHTGTTNQRSPPGERTR | 59 |
| GHTGTTNQRSPPGERTRGR | 60 |
| GQRRWSTR | 61 |
| GRQPGDYDDDR | 62 |
| GRQPGDYDDDRR | 63 |
| GRQPGDYDDDRRQPR | 64 |
| GRQPGDYDDDRRQPR | 65 |
| GRREEEEDEDEEEEGSNR | 66 |
| GRREEEEDEDEEEEGSNREVR | 67 |
| GSEEEGDITNPINLR | 68 |
| GSEEEGDITNPINLREGEPDLSNNFGK | 69 |
| GTGNLELVAVR | 70 |
| GTGNLELVAVRK | 71 |
| HADADNILVIQQGQATVTVANGNNR | 72 |
| HADADNILVIQQGQATVTVANGNNRK | 73 |
| HADADNILVIQQGQATVTVANGNNRKSFNLDEGHALR | 74 |
| HAKSVSKK | 75 |
| HAKSVSKKGSEEEGDITNPINLR | 76 |
| HDNQNLR | 77 |
| HDNQNLRVAKISMPVNTPGQFEDFFPASSR | 78 |
| IFLAGDKDNVIDQIEK | 79 |
| IFLAGDKDNVIDQIEKQAK | 80 |
| IFLAGDKDNVIDQIEKQAKDLAFPGSGEQVEK | 81 |
| IPSGFISYILNR | 82 |
| IPSGFISYILNRHDNQNLR | 83 |
| IPSGFISYILNRHDNQNLRVAK | 84 |
| IPSGFISYILNRHDNQNLRVAKISMPVNTPGQFEDFFPASSR | 85 |
| IRPEGREGEQEWGTPGSHVREETSR | 86 |
| IRPEGREGEQEWGTPGSHVREETSRNNPFYFPSR | 87 |
| IRVLQRFDQR | 88 |
| ISMPVNTPGQFEDFFPASSR | 89 |
| ISMPVNTPGQFEDFFPASSRDQSSYLQGFSR | 90 |
| ISMPVNTPGQFEDFFPASSRDQSSYLQGFSRNTLEAAFNAEF | 91 |
| NEIR | |
| ISMPVNTPGQFEDFFPASSRDQSSYLQGFSRNTLEAAFNAEF | 92 |
| NEIRR | |
| IVQIEAKPNTLVLPK | 93 |
| KEQQQR | 94 |
| KEQQQRGRR | 95 |
| KGSEEEGDITNPINLR | 96 |
| KGSEEEGDITNPINLREGEPDLSNNFGK | 97 |
| KGSEEEGDITNPINLREGEPDLSNNFGKLFEVKPDK | 98 |
| KIRPEGREGEQEWGTPGSHVREETSR | 99 |
| KNPQLQDLDMMLTCVEIK | 100 |
| KNPQLQDLDMMLTCVEIKEGALMLPHFNSK | 101 |
| KNPQLQDLDMMLTCVEIKEGALMLPHFNSKAMVIVVVNK | 102 |
| KSFNLDEGHALR | 103 |
| KSFNLDEGHALRIPSGFISYILNR | 104 |
| KSFNLDEGHALRIPSGFISYILNRHDNQNLR | 105 |
| KTENPCAQR | 106 |
| KTENPCAQRCLQSCQQEPDDLK | 107 |
| KTENPCAQRCLQSCQQEPDDLKQK | 108 |
| LEYDPR | 109 |
| LEYDPRCVYDPR | 110 |
| LEYDPRCVYDPRGHTGTTNQR | 111 |
| LEYDPRCVYDPRGHTGTTNQRSPPGER | 112 |
| LFEVKPDK | 113 |
| LFEVKPDKK | 114 |
| LFEVKPDKKNPQLQDLDMMLTCVEIK | 115 |
| LFEVKPDKKNPQLQDLDMMLTCVEIKEGALMLPHFNSK | 116 |
| LIKNQKESHFVSARPQSQSQSPSSPEK | 117 |
| NNPFYFPSR | 118 |
| NNPFYFPSRR | 119 |
| NNPFYFPSRRFSTR | 120 |
| NNPFYFPSRRFSTRYGNQNGR | 121 |
| NPQLQDLDMMLTCVEIK | 122 |
| NPQLQDLDMMLTCVEIKEGALMLPHFNSK | 123 |
| NPQLQDLDMMLTCVEIKEGALMLPHFNSKAMVIVVVNK | 124 |
| NQKESHFVSARPQSQSQSPSSPEK | 125 |
| NQKESHFVSARPQSQSQSPSSPEKESPEKEDQEEENQGGK | 126 |
| NTLEAAFNAEFNEIR | 127 |
| NTLEAAFNAEFNEIRR | 128 |
| NTLEAAFNAEFNEIRRVLLEENAGGEQEER | 129 |
| NTLEAAFNAEFNEIRRVLLEENAGGEQEERGQR | 130 |
| QAKDLAFPGSGEQVEK | 131 |
| QFQNLQNHR | 132 |
| QFQNLQNHRIVQIEAKPNTLVLPKHADADNILVIQQGQATVT | 133 |
| VANGNNRK | |
| QKACESR | 134 |
| QKACESRCTKLEYDPR | 135 |
| QPGDYDDDRR | 136 |
| QPGDYDDDRRQPR | 137 |
| QPGDYDDDRRQPRR | 138 |
| QPREDWRRPSHQQPR | 139 |
| QPREDWRRPSHQQPRK | 140 |
| QPRREEGGR | 141 |
| QPRREEGGRWGPAGPR | 142 |
| REEEEDEDEEEEGSNR | 143 |
| REEEEDEDEEEEGSNREVR | 144 |
| REEEEDEDEEEEGSNREVRR | 145 |
| REEGGR | 146 |
| REEGGRWGPAGPR | 147 |
| REEGGRWGPAGPRER | 148 |
| RFSTRYGNQNGR | 149 |
| RFSTRYGNQNGRIR | 150 |
| RPSHQQPR | 151 |
| RVLLEENAGGEQEER | 152 |
| RVLLEENAGGEQEERGQR | 153 |
| RWSTRSSENNEGVIVK | 154 |
| SFNLDEGHALR | 155 |
| SFNLDEGHALRIPSGFISYILNR | 156 |
| SFNLDEGHALRIPSGFISYILNRHDNQNLR | 157 |
| SPPGERTRGR | 158 |
| SPPGERTRGRQPGDYDDDR | 159 |
| SRQFQNLQNHR | 160 |
| SRQFQNLQNHRIVQIEAKPNTLVLPKHADADNILVIQQGQAT | 161 |
| VTVANGNNR | |
| SSENNEGVIVK | 162 |
| SSENNEGVIVKVSK | 164 |
| SSENNEGVIVKVSKEHVEELTK | 164 |
| SSPYQKK | 165 |
| SSPYQKKTENPCAQR | 166 |
| SSPYQKKTENPCAQRCLQSCQQEPDDLK | 167 |
| SVSKKGSEEEGDITNPINLR | 168 |
| TENPCAQR | 169 |
| TENPCAQRCLQSCQQEPDDLK | 170 |
| TENPCAQRCLQSCQQEPDDLKQK | 171 |
| TENPCAQRCLQSCQQEPDDLKQKACESR | 172 |
| TRGRQPGDYDDDR | 173 |
| TRGRQPGDYDDDRR | 174 |
| VAKISMPVNTPGQFEDFFPASSR | 175 |
| VAKISMPVNTPGQFEDFFPASSRDQSSYLQGFSRNTLEAAFN | 176 |
| AEFNEIR | |
| VLLEENAGGEQEER | 177 |
| VLLEENAGGEQEERGQR | 178 |
| VLLEENAGGEQEERGQRR | 179 |
| VLLEENAGGEQEERGQRRWSTR | 180 |
| VLQRFDQRSRQFQNLQNHR | 181 |
| VSKEHVEELTK | 182 |
| VSPLMLLLGILVLASVSATHAK | 183 |
| WGPAGPR | 184 |
| WGPAGPRER | 185 |
| WGPAGPRERER | 186 |
| WSTRSSENNEGVIVK | 187 |
| WSTRSSENNEGVIVKVSKEHVEELTK | 188 |
| YGNQNGR | 189 |
| YGNQNGRIRVLQR | 190 |
| YGNQNGRIRVLQRFDQR | 191 |
| YTARLKEGDVFIMPAAHPVAINASSELHLLGFGINAENNHRI | 192 |
| FLAGDK | |

TABLE 20
Ara h1 Sequences Identified in Prepared Peanut
Extract

| | # | |
|---|---|---|
| | Appearance | |
| | in 7 | UniProt |
| Ara h1 Peptide | UniProt | Reference |
| Sequence | References | Nos |
| DLAFPGSGEQVEK | 7 | P43238, |
| (SEQ ID NO: 17) | | P43237, |
| | | E5G076, |
| | | Q6PSU3, |
| | | Q6PSU6, |
| | | Q6PSU5, |
| | | Q6PSU4 |
| GTGNLELVAVR | 7 | P43238, |
| (SEQ ID NO: 70) | | P43237, |
| | | E5G076, |
| | | Q6PSU3, |
| | | Q6PSU6, |
| | | Q6PSU5, |
| | | Q6PSU4 |
| VLLEENAGGEQEER | 7 | P43238, |
| (SEQ ID NO: 177) | | P43237, |
| | | E5G076, |
| | | Q6PSU3, |
| | | Q6PSU6, |
| | | Q6PSU5, |
| | | Q6PSU4 |
| DQSSYLQGFSR | 5 | P43238, |
| (SEQ ID NO: 20) | | P43237, |
| | | E5G076, |
| | | Q6PSU3, |
| | | Q6PSU4 |
| HADADNILVIQQGQATVTVANGNNR | 5 | P43238, |
| (SEQ ID NO: 72) | | P43237, |
| | | E5G076, |
| | | Q6PSU3, |
| | | Q6PSU4 |
| NTLEAAFNAEFNEIR | 5 | P43238, |
| (SEQ ID NO: 127) | | P43237, |
| | | E5G076, |
| | | Q6PSU3, |
| | | Q6PSU4 |
| EQEWEEEEEDEEEEGSNR | 4 | P43237, |
| (SEQ ID NO: 193) | | E5G076, |
| | | Q6PSU3, |
| | | Q6PSU6 |
| IPSGFISYILNR | 4 | P43238, |
| (SEQ ID NO: 82) | | P43237, |
| | | Q6PSU3, |
| | | Q6PSU4 |
| NNPFYFPSR | 4 | P43238, |
| (SEQ ID NO: 118) | | P43237, |
| | | E5G076, |
| | | Q6PSU3 |
| SFNLDEGHALR | 4 | P43238, |
| (SEQ ID NO: 155) | | P43237, |
| | | Q6PSU3, |
| | | Q6PSU4 |
| GSEEEDITNPINLR | 3 | P43237, |
| (SEQ ID NO: 194) | | Q6PSU3, |
| | | Q6PSU6 |
| IVQIEAKPNTLVLPK | 3 | P43238, |
| (SEQ ID NO: 93) | | E5G076, |
| | | Q6PSU4 |
| EGEQEWGTPGSEVR | 2 | P43237, |
| (SEQ ID NO: 195) | | Q6PSU3 |
| EGEQEWGTPGSHVR | 2 | P43238, |
| (SEQ ID NO: 40) | | E5G076 |
| IVQIEARPNTLVLPK | 2 | P43237, |
| (SEQ ID NO: 196) | | Q6PSU3 |

TABLE 21
Ara h2 Sequences Identified in Prepared
Peanut Extract

| | # | |
|---|---|---|
| | Appearance | |
| Ara h2 | in 1 | UniProt |
| Peptide | UniProt | Reference |
| Sequence | References | Nos |
| GAGSSQHQER | 1 | Q6PSU2 |
| (SEQ ID NO: 197) | | |
| QQEQQFK | 1 | Q6PSU2 |
| (SEQ ID NO: 198) | | |

TABLE 22
Ara h3 Sequences Identified in Prepared
Peanut Extract

| | # | |
|---|---|---|
| | Appearance | |
| | in 16 | UniProt |
| Ara h3 | UniProt | Reference |
| Peptide Sequence | References | Nos |
| RPFYSNAPQEIFIQQGR | 12 | Q5I6T2, |
| (SEQ ID NO: 199) | | B5TYU1, |
| | | Q9FZ11, |
| | | A1DZF1, |
| | | Q82580, |
| | | Q8LL03, |
| | | Q9SQH7, |
| | | Q647H4, |
| | | Q6T2T4, |
| | | Q8LKN1, |
| | | Q647H3, |
| | | A1DZF0 |
| AHVQVVDSNGNR | 10 | Q5I6T2, |
| (SEQ ID NO: 200) | | B5TYU1, |
| | | Q9FZ11, |
| | | O82580, |
| | | Q0GM57, |
| | | E5G077, |
| | | Q9SQH7, |
| | | Q647H3, |
| | | Q6IWG5, |
| | | A1DZF0 |
| FNLAGNHEQEFLR | 10 | Q5I6T2, |
| (SEQ ID NO: 201) | | B5TYU1, |
| | | Q9FZ11, |
| | | Q8LL03, |
| | | Q9SQH7, |
| | | Q647H4, |
| | | Q6T2T4, |
| | | Q8LKN1, |
| | | Q647H3, |
| | | A1DZF0 |
| NALFVPHYNTNAHSIIYALR | 9 | Q5I6T2 (3x), |
| (SEQ ID NO: 202) | | B5TYU1 (3x), |
| | | Q9FZ11 (3x), |
| | | Q9SQH7 (3x), |
| | | Q647H4 (3x), |
| | | Q6T2T4 (3x), |
| | | Q8LKN1 (3x), |
| | | Q647H3 (3x), |
| | | A1DZF0 (3x) |
| SPDIYNPQAGSLK | 9 | Q5I6T2, |
| (SEQ ID NO: 203) | | B5TYU1, |
| | | Q9FZ11, |
| | | O82580, |
| | | Q647H4, |
| | | Q6T2T4, |
| | | Q8LKN1, |
| | | Q647H3, |
| | | A1DZF0 |
| WLGLSAEYGNLYR | 9 | Q5I6T2, |
| (SEQ ID NO: 204) | | B5TYU1, |
| | | Q9FZ11, |
| | | Q9SQH7, |
| | | Q647H4, |
| | | Q6T2T4, |
| | | Q8LKN1, |
| | | Q647H3, |
| | | A1DZF0 |
| VYDEELQEGHVLVVPQNFAVAGK | 7 | Q5I6T2, |
| (SEQ ID NO: 205) | | B5TYU1, |
| | | Q9FZ11, |
| | | O82580, |
| | | Q9SQH7, |
| | | Q647H3, |
| | | A1DZF0 |
| SQSENFEYVAFK | 6 | O82580 (3x), |
| (SEQ ID NO: 206) | | Q9SQH7 (3x), |
| | | Q647H4 (3x), |
| | | Q6T2T4 (3x), |
| | | Q8LKN1 (3x), |
| | | A1DZF0 (3x) |
| AGQEQENEGGNIFSGFTPEFLAQA | 4 | Q647H4 (3x), |
| FQVDDR | | Q6T2T4 (3x), |
| (SEQ ID N0: 207) | | Q8LKN1 (3x), |
| | | Q647H3 (3x) |
| GENESDEQGAIVTVR | 4 | Q647H4, |
| (SEQ ID NO: 208) | | Q6T2T4, |
| | | Q8LKN1, |
| | | Q647H3 |
| QQYERPDEEEEYDEDEYEYDEEER | 4 | Q647H4, |
| (SEQ ID NO: 209) | | Q6T2T4, |
| | | Q8LKN1, |
| | | Q647H3 |
| SQSDNFEYVAFK | 4 | Q5I6T2, |
| (SEQ ID NO: 210) | | B5TYU1, |
| | | Q9FZ11, |
| | | Q647H3 |
| TANDLNLLILR | 4 | Q5I6T2, |
| (SEQ ID NO: 211) | | Q9FZ11, |
| | | O82580, |
| | | Q8LKN1 |
| TANELNLLILR | 4 | B5TYU1 (3x), |
| (SEQ ID NO: 212) | | Q647H4 (3x), |
| | | Q6T2T4 (3x), |
| | | A1DZF0 (3x) |
| TDSRPSIANLAGENSFIDNLPEEV | 4 | Q647H4 (3x), |
| VANSYGLPR | | Q6T2T4 (3x), |
| (SEQ ID NO: 213) | | Q8LKN1 (3x), |
| | | A1DZF0 (3x) |
| AHVQVVDSNGDR | 3 | Q647H4, |
| (SEQ ID NO: 214) | | Q6T2T4, |
| | | Q8LKN1 |
| AQSENYEYLAFK | 3 | Q0GM57, |
| (SEQ ID NO: 215) | | E5G077, |
| | | Q6IWG5 |
| FNEGDLIAVPTGVAFWLYNDHDTD | 3 | B5TYU1, |
| VVAVSLTDTNNNDNQLDQFPR | | Q9SQH7, |
| (SEQ ID NO: 216) | | A1DZF0 |
| GADEEEEYDEDEYEYDEEDR | 3 | Q5I6T2, |
| (SEQ ID NO: 217) | | B5TYU1, |
| | | Q9FZ11 |
| SSNPDIYNPQAGSLR | 3 | Q0GM57, |
| (SEQ ID NO: 218) | | E5G077, |
| | | Q6IWG5 |
| SVNELDLPILGWLGLSAQHGTIYR | 3 | Q0GM57, |
| (SEQ ID NO: 219) | | E5G077, |
| | | Q6IWG5 |
| VFDEELQEGHVLVVPQNFAVAGK | 3 | Q647H4, |
| (SEQ ID NO: 220) | | Q6T2T4, |
| | | Q8LKN1 |
| VYDEELQEGHVLVVPQNFAVAAK | 3 | Q0GM57, |
| (SEQ ID NO: 221) | | E5G077, |
| | | Q6IWG5 |
| AGQEEENEGGNIFSGFTPEFLAQA | 2 | B5TYU1, |
| FQVDDR | | Q9FZ11 |
| (SEQ ID NO: 222) | | |
| AGQEEENEGGNIFSGFTPEFLEQA | 2 | Q5I6T2, |
| FQVDDR | | O82580 |
| (SEQ ID NO: 223) | | |
| FFVPPFQQSPR | 2 | Q9SQH7, |
| (SEQ ID NO: 224) | | A1DZF0 |
| TDSRPSIANLAGENSIIDNLPEEV | 2 | Q0GM57 (3x), |
| VANSYR | | Q6IWG5 (3x) |
| (SEQ ID NO: 225) | | |
| AGQEEEDEGGNIFSGFTPEFLEQA | 1 | Q9SQH7 |
| FQVDDR | | |
| (SEQ ID NO: 226) | | |
| AGQEQENEGGNIFSGFTSEFLAQA | 1 | A1DZF0 |
| FQVDDR | | |
| (SEQ ID NO: 227) | | |
| GENESEEEGAIVTVK | 1 | Q9FZ11 |
| (SEQ ID NO: 228) | | |
| GENESEEQGAIVTVK | 1 | A1DZF0 (3x) |
| (SEQ ID NO: 229) | | |
| SPDEEEEYDEDEYAEEER | 1 | A1DZF0 |
| (SEQ ID NO: 230) | | |
| SQSEHFLYVAFK | 1 | Q647H2 |
| (SEQ ID NO: 231) | | |
| TDSRPSIANLAGENSIIDNLPEEV | 1 | Q647H3 (3x) |
| VANSYGLPR | | |
| (SEQ ID NO: 232) | | |
| TDSRPSIANLAGENSVIDNLPEEV | 1 | B5TYU1 |
| VANSYGLPR | | |
| (SEQ ID NO: 233) | | |
| TDSRPSIANQAGENSIIDNLPEEV | 1 | E5G077 |
| VANSYR | | |
| (SEQ ID NO: 234) | | |
| VFDEELQEGQSLVVPQNFAVAAK | 1 | Q647H2 |
| (SEQ ID NO: 235) | | |

TABLE 23
Ara h6 Sequences Identified in Prepared
Peanut Extract

| | # | |
|---|---|---|
| | Appearance | |
| Ara h6 | in 2 | UniProt |
| Peptide | UniProt | Reference |
| Sequence | References | Nos |
| IMGEQEQYDSYDIR | 2 | Q647G9, |
| (SEQ ID NO: 236) | | A1DZE9 |
| QMVQQFK | 1 | Q647G9 |
| (SEQ ID NO: 237) | | |

### Equivalents

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A method for quantifying peanut allergens in composition, the method comprising:
contacting a composition comprising one or more peanut allergens with an aqueous extract medium to produce an aqueous peanut extract;
digesting the one or more peanut protein allergens present in the aqueous peanut extract to generate peanut protein allergen digest products;
fragmenting the one or more peanut protein allergen digest products to generate peptide fragments;
analyzing the peptide fragments and/or the peanut protein allergen digest products using a mass spectroscopic method to generate a peanut allergen signature; and
determining an amount of the one or more peanut protein allergens in the composition by comparing the peanut allergen signature to a calibration curve,
wherein the peanut allergen signature comprises allergen digest products from Ara h1, Ara h2 and Ara h6, and
wherein the one or more peanut protein allergens in the aqueous peanut extract is present at a concentration of less than about 2 μg/ml.

2-5. (cancel)

6. The method of claim 1, wherein the allergen digest products are between about 4 amino acids and about 50 amino acids in length.

7. The method of claim 1, wherein the allergen digest products are between about 6 amino acids and about 30 amino acids in length.

8. The method of claim 1, wherein the allergen digest products are between about 15 amino acids and about 20 amino acids in length.

9. The method of claim 1, wherein the mass spectrographic method is selected from the group consisting of one or any combination of LC-MS, LC-MS-MS, nano-LC-MS-MS, and nanoHPLC-MS-MS.

10. The method of claim 1, wherein the signature comprises Ara h1 allergen digest products having an amino acid sequence selected from the group consisting of SEQ ID NO:17, SEQ ID NO:70, SEQ ID NO: 177, SEQ ID NO:155, SEQ ID NO:93 and SEQ ID NO:40.

11-12. (cancel)

13. The method of claim 12, wherein the signature comprises allergen digest products having amino acid sequences selected from the group consisting of SEQ ID NO:17, SEQ ID NO:70, SEQ ID NO:177, SEQ ID NO:197, SEQ ID NO:198, SEQ ID NO:199, SEQ ID NO:200, SEQ ID NO:201, SEQ ID NO:236 and SEQ ID NO:237.

14-18. (cancel)

19. The method of claim 1, wherein the peanut allergens are digested with one or more proteases selected from the group consisting of trypsin, endoproteinase Lys-C and endoproteinase Arg-C.

20. (cancel)

21. The method of claim 1, wherein the composition comprising one or more peanut allergens comprises an internal standard that comprises one or more heavy isotopes.

22. The method of claim 1, wherein the signature comprises allergen digest products that do not contain missed proteolytic cleavage sites.

23-47. (cancel)

48. The method of claim 1, wherein the aqueous peanut extract is made from peanut flour, roasted peanuts or raw peanuts.

49. The method of claim 1, wherein the one or more peanut protein allergens in the aqueous peanut extract is present at a concentration of less than about 1.0 μg/ml.

50. The method of claim 1, wherein the one or more peanut protein allergens in the aqueous peanut extract is present at a concentration of less than about 0.5 μg/ml.

51. The method of claim 1, wherein the peanut allergen signature comprises allergen digest products that are present in a majority of isoforms of Ara h1, Ara h2 and Ara h6.

52. The method of claim 50, wherein the peanut allergen signature comprises allergen digest products from Ara h1, Ara h2 and Ara h6 having amino acid sequences selected from the group consisting of SEQ ID NO:17, SEQ ID NO:70, SEQ ID NO:177, SEQ ID NO: 197, SEQ ID NO:198, SEQ ID NO:199, SEQ ID NO:200, SEQ ID NO:201, SEQ ID NO:236 and SEQ ID NO:237.

53. The method of claim 1, wherein the one or more peanut protein allergen digest products are between about 6 amino acids and about 30 amino acids in length.

54. The method of claim 1, wherein the one or more peanut protein allergen digest products are between about 15 amino acids and about 20 amino acids in length.

55. A method for quantifying one or more peanut protein allergens in a composition, the method comprising:
contacting a composition comprising one or more peanut allergens with an aqueous extract medium to produce an aqueous peanut extract;
digesting the one or more peanut protein allergens present in the aqueous peanut extract to generate one or more peanut protein allergen digest products;
fragmenting the one or more peanut protein allergen digest products using a mass spectroscopic method to generate peptide fragments;
analyzing the peptide fragments and/or the one or more peanut protein allergen digest products to generate a peanut allergen signature comprising allergen digest products from Ara h1, Ara h2 and Ara h6; and
determining an amount of the one or more peanut protein allergens in the composition by comparing the peanut allergen signature to a calibration curve.

56. A method for quantifying one or more peanut protein allergens in a composition, the method comprising:
contacting a composition comprising one or more peanut allergens with an aqueous extract medium to produce an aqueous peanut extract;
digesting the one or more peanut protein allergens present in the aqueous peanut extract to generate one or more peanut protein allergen digest products;
fragmenting the one or more peanut protein allergen digest products to generate peptide fragments;
analyzing the peptide fragments and/or the one or more peanut protein allergen digest products using a mass spectroscopic method to generate a peanut allergen signature comprising allergen digest products from Ara h1, Ara h2 and Ara h6; and
determining an amount of the one or more peanut protein allergens in the composition by comparing the peanut allergen signature to a calibration curve,
wherein the one or more peanut protein allergens in the aqueous peanut extract is present at a concentration of less than about 1.5 μg/ml.


SEQUENCE LISTING (253 sequences)

SEQ ID NO: 1 - protein, 14 aa, Arachis hypogaea
ACESRCTKLEYDPR

SEQ ID NO: 2 - protein, 20 aa, Arachis hypogaea
ACESRCTKLEYDPRCVYDPR

SEQ ID NO: 3 - protein, 9 aa, Arachis hypogaea
AMVIVVVNK

SEQ ID NO: 4 - protein, 20 aa, Arachis hypogaea
AMVIVVVNKGTGNLELVAVR

SEQ ID NO: 5 - protein, 21 aa, Arachis hypogaea
AMVIVVVNKGTGNLELVAVRK

SEQ ID NO: 6 - protein, 13 aa, Arachis hypogaea
CLQSCQQEPDDLK

SEQ ID NO: 7 - protein, 15 aa, Arachis hypogaea
CLQSCQQEPDDLKQK

SEQ ID NO: 8 - protein, 20 aa, Arachis hypogaea
CLQSCQQEPDDLKQKACESR

SEQ ID NO: 9 - protein, 23 aa, Arachis hypogaea
CLQSCQQEPDDLKQKACESRCTK

SEQ ID NO: 10 - protein, 9 aa, Arachis hypogaea
CTKLEYDPR

SEQ ID NO: 11 - protein, 15 aa, Arachis hypogaea
CTKLEYDPRCVYDPR

SEQ ID NO: 12 - protein, 24 aa, Arachis hypogaea
CTKLEYDPRCVYDPRGHTGTTNQR

SEQ ID NO: 13 - protein, 6 aa, Arachis hypogaea
CVYDPR

SEQ ID NO: 14 - protein, 15 aa, Arachis hypogaea
CVYDPRGHTGTTNQR

SEQ ID NO: 15 - protein, 21 aa, Arachis hypogaea
CVYDPRGHTGTTNQRSPPGER

SEQ ID NO: 16 - protein, 23 aa, Arachis hypogaea
CVYDPRGHTGTTNQRSPPGERTR

SEQ ID NO: 17 - protein, 13 aa, Arachis hypogaea
DLAFPGSGEQVEK

SEQ ID NO: 18 - protein, 16 aa, Arachis hypogaea
DLAFPGSGEQVEKLIK

SEQ ID NO: 19 - protein, 12 aa, Arachis hypogaea
DNVIDQIEKQAK

SEQ ID NO: 20 - protein, 11 aa, Arachis hypogaea
DQSSYLQGFSR

SEQ ID NO: 21 - protein, 27 aa, Arachis hypogaea
DQSSYLQGFSRNTLEAAFNAEFNEIRR

SEQ ID NO: 22 - protein, 19 aa, Arachis hypogaea
EDQEEENQGGKGPLLSILK

SEQ ID NO: 23 - protein, 12 aa, Arachis hypogaea
EDWRRPSHQQPR

SEQ ID NO: 24 - protein, 12 aa, Arachis hypogaea
EEDWRQPREDWR

SEQ ID NO: 25 - protein, 20 aa, Arachis hypogaea
EEDWRQPREDWRRPSHQQPR

SEQ ID NO: 26 - protein, 23 aa, Arachis hypogaea
EEEEDEDEEEEGSNREVRRYTAR

SEQ ID NO: 27 - protein, 12 aa, Arachis hypogaea
EEGGRWGPAGPR

SEQ ID NO: 28 - protein, 14 aa, Arachis hypogaea
EEGGRWGPAGPRER

SEQ ID NO: 29 - protein, 16 aa, Arachis hypogaea
EEGGRWGPAGPRERER

SEQ ID NO: 30 - protein, 14 aa, Arachis hypogaea
EETSRNNPFYFPSR

SEQ ID NO: 31 - protein, 15 aa, Arachis hypogaea
EETSRNNPFYFPSRR

SEQ ID NO: 32 - protein, 19 aa, Arachis hypogaea
EETSRNNPFYFPSRRFSTR

SEQ ID NO: 33 - protein, 12 aa, Arachis hypogaea
EGALMLPHFNSK

SEQ ID NO: 34 - protein, 21 aa, Arachis hypogaea
EGALMLPHFNSKAMVIVVVNK

SEQ ID NO: 35 - protein, 42 aa, Arachis hypogaea
EGDVFIMPAAHPVAINASSELHLLGFGINAENNHRIFLAGDK

SEQ ID NO: 36 - protein, 51 aa, Arachis hypogaea
EGDVFIMPAAHPVAINASSELHLLGFGINAENNHRIFLAGDKDNVIDQIEK

SEQ ID NO: 37 - protein, 12 aa, Arachis hypogaea
EGEPDLSNNFGK

SEQ ID NO: 38 - protein, 21 aa, Arachis hypogaea
EGEPDLSNNFGKLFEVKPDKK

SEQ ID NO: 39 - protein, 38 aa, Arachis hypogaea
EGEPDLSNNFGKLFEVKPDKKNPQLQDLDMMLTCVEIK

SEQ ID NO: 40 - protein, 14 aa, Arachis hypogaea
EGEQEWGTPGSHVR

SEQ ID NO: 41 - protein, 29 aa, Arachis hypogaea
EGEQEWGTPGSHVREETSRNNPFYFPSRR

SEQ ID NO: 42 - protein, 8 aa, Arachis hypogaea
EHVEELTK

SEQ ID NO: 43 - protein, 11 aa, Arachis hypogaea
EHVEELTKHAK

SEQ ID NO: 44 - protein, 8 aa, Arachis hypogaea
EQQQRGRR

SEQ ID NO: 45 - protein, 23 aa, Arachis hypogaea
EQQQRGRREEEEDEDEEEEGSNR

SEQ ID NO: 46 - protein, 7 aa, Arachis hypogaea
EREEDWR

SEQ ID NO: 47 - protein, 10 aa, Arachis hypogaea
EREEDWRQPR

SEQ ID NO: 48 - protein, 9 aa, Arachis hypogaea
EREREEDWR

SEQ ID NO: 49 - protein, 12 aa, Arachis hypogaea
EREREEDWRQPR

SEQ ID NO: 50 - protein, 21 aa, Arachis hypogaea
ESHFVSARPQSQSQSPSSPEK

SEQ ID NO: 51 - protein, 37 aa, Arachis hypogaea
ESHFVSARPQSQSQSPSSPEKESPEKEDQEEENQGGK

SEQ ID NO: 52 - protein, 16 aa, Arachis hypogaea
ESPEKEDQEEENQGGK

SEQ ID NO: 53 - protein, 8 aa, Arachis hypogaea
EVRRYTAR

SEQ ID NO: 54 - protein, 6 aa, Arachis hypogaea
FDQRSR

SEQ ID NO: 55 - protein, 15 aa, Arachis hypogaea
FDQRSRQFQNLQNHR

SEQ ID NO: 56 - protein, 11 aa, Arachis hypogaea
FSTRYGNQNGR

SEQ ID NO: 57 - protein, 13 aa, Arachis hypogaea
FSTRYGNQNGRIR

SEQ ID NO: 58 - protein, 13 aa, Arachis hypogaea
FSTRYGNQNGRIR

SEQ ID NO: 59 - protein, 17 aa, Arachis hypogaea
GHTGTTNQRSPPGERTR

SEQ ID NO: 60 - protein, 19 aa, Arachis hypogaea
GHTGTTNQRSPPGERTRGR

SEQ ID NO: 61 - protein, 8 aa, Arachis hypogaea
GQRRWSTR

SEQ ID NO: 62 - protein, 11 aa, Arachis hypogaea
GRQPGDYDDDR

SEQ ID NO: 63 - protein, 12 aa, Arachis hypogaea
GRQPGDYDDDRR

SEQ ID NO: 64 - protein, 15 aa, Arachis hypogaea
GRQPGDYDDDRRQPR

SEQ ID NO: 65 - protein, 15 aa, Arachis hypogaea
GRQPGDYDDDRRQPR

SEQ ID NO: 66 - protein, 18 aa, Arachis hypogaea
GRREEEEDEDEEEEGSNR

SEQ ID NO: 67 - protein, 21 aa, Arachis hypogaea
GRREEEEDEDEEEEGSNREVR

SEQ ID NO: 68 - protein, 15 aa, Arachis hypogaea
GSEEEGDITNPINLR

SEQ ID NO: 69 - protein, 27 aa, Arachis hypogaea
GSEEEGDITNPINLREGEPDLSNNFGK

SEQ ID NO: 70 - protein, 11 aa, Arachis hypogaea
GTGNLELVAVR

SEQ ID NO: 71 - protein, 12 aa, Arachis hypogaea
GTGNLELVAVRK

SEQ ID NO: 72 - protein, 25 aa, Arachis hypogaea
HADADNILVIQQGQATVTVANGNNR

SEQ ID NO: 73 - protein, 26 aa, Arachis hypogaea
HADADNILVIQQGQATVTVANGNNRK

SEQ ID NO: 74 - protein, 37 aa, Arachis hypogaea
HADADNILVIQQGQATVTVANGNNRKSFNLDEGHALR

SEQ ID NO: 75 - protein, 8 aa, Arachis hypogaea
HAKSVSKK

SEQ ID NO: 76 - protein, 23 aa, Arachis hypogaea
HAKSVSKKGSEEEGDITNPINLR

SEQ ID NO: 77 - protein, 7 aa, Arachis hypogaea
HDNQNLR

SEQ ID NO: 78 - protein, 30 aa, Arachis hypogaea
HDNQNLRVAKISMPVNTPGQFEDFFPASSR

SEQ ID NO: 79 - protein, 16 aa, Arachis hypogaea
IFLAGDKDNVIDQIEK

SEQ ID NO: 80 - protein, 19 aa, Arachis hypogaea
IFLAGDKDNVIDQIEKQAK

SEQ ID NO: 81 - protein, 32 aa, Arachis hypogaea
IFLAGDKDNVIDQIEKQAKDLAFPGSGEQVEK

SEQ ID NO: 82 - protein, 12 aa, Arachis hypogaea
IPSGFISYILNR

SEQ ID NO: 83 - protein, 19 aa, Arachis hypogaea
IPSGFISYILNRHDNQNLR

SEQ ID NO: 84 - protein, 22 aa, Arachis hypogaea
IPSGFISYILNRHDNQNLRVAK

SEQ ID NO: 85 - protein, 42 aa, Arachis hypogaea
IPSGFISYILNRHDNQNLRVAKISMPVNTPGQFEDFFPASSR

SEQ ID NO: 86 - protein, 25 aa, Arachis hypogaea
IRPEGREGEQEWGTPGSHVREETSR

SEQ ID NO: 87 - protein, 34 aa, Arachis hypogaea
IRPEGREGEQEWGTPGSHVREETSRNNPFYFPSR

SEQ ID NO: 88 - protein, 10 aa, Arachis hypogaea
IRVLQRFDQR

SEQ ID NO: 89 - protein, 20 aa, Arachis hypogaea
ISMPVNTPGQFEDFFPASSR

SEQ ID NO: 90 - protein, 31 aa, Arachis hypogaea
ISMPVNTPGQFEDFFPASSRDQSSYLQGFSR

SEQ ID NO: 91 - protein, 46 aa, Arachis hypogaea
ISMPVNTPGQFEDFFPASSRDQSSYLQGFSRNTLEAAFNAEFNEIR

SEQ ID NO: 92 - protein, 47 aa, Arachis hypogaea
ISMPVNTPGQFEDFFPASSRDQSSYLQGFSRNTLEAAFNAEFNEIRR

SEQ ID NO: 93 - protein, 15 aa, Arachis hypogaea
IVQIEAKPNTLVLPK

SEQ ID NO: 94 - protein, 6 aa, Arachis hypogaea
KEQQQR

SEQ ID NO: 95 - protein, 9 aa, Arachis hypogaea
KEQQQRGRR

SEQ ID NO: 96 - protein, 16 aa, Arachis hypogaea
KGSEEEGDITNPINLR

SEQ ID NO: 97 - protein, 28 aa, Arachis hypogaea
KGSEEEGDITNPINLREGEPDLSNNFGK

SEQ ID NO: 98 - protein, 36 aa, Arachis hypogaea
KGSEEEGDITNPINLREGEPDLSNNFGKLFEVKPDK

SEQ ID NO: 99 - protein, 26 aa, Arachis hypogaea
KIRPEGREGEQEWGTPGSHVREETSR

SEQ ID NO: 100 - protein, 18 aa, Arachis hypogaea
KNPQLQDLDMMLTCVEIK

SEQ ID NO: 101 - protein, 30 aa, Arachis hypogaea
KNPQLQDLDMMLTCVEIKEGALMLPHFNSK

SEQ ID NO: 102 - protein, 39 aa, Arachis hypogaea
KNPQLQDLDMMLTCVEIKEGALMLPHFNSKAMVIVVVNK

SEQ ID NO: 103 - protein, 12 aa, Arachis hypogaea
KSFNLDEGHALR

SEQ ID NO: 104 - protein, 24 aa, Arachis hypogaea
KSFNLDEGHALRIPSGFISYILNR

SEQ ID NO: 105 - protein, 31 aa, Arachis hypogaea
KSFNLDEGHALRIPSGFISYILNRHDNQNLR

SEQ ID NO: 106 - protein, 9 aa, Arachis hypogaea
KTENPCAQR

SEQ ID NO: 107 - protein, 22 aa, Arachis hypogaea
KTENPCAQRCLQSCQQEPDDLK

SEQ ID NO: 108 - protein, 24 aa, Arachis hypogaea
KTENPCAQRCLQSCQQEPDDLKQK

SEQ ID NO: 109 - protein, 6 aa, Arachis hypogaea
LEYDPR

SEQ ID NO: 110 - protein, 12 aa, Arachis hypogaea
LEYDPRCVYDPR

SEQ ID NO: 111 - protein, 21 aa, Arachis hypogaea
LEYDPRCVYDPRGHTGTTNQR

SEQ ID NO: 112 - protein, 27 aa, Arachis hypogaea
LEYDPRCVYDPRGHTGTTNQRSPPGER

SEQ ID NO: 113 - protein, 8 aa, Arachis hypogaea
LFEVKPDK

SEQ ID NO: 114 - protein, 9 aa, Arachis hypogaea
LFEVKPDKK

SEQ ID NO: 115 - protein, 26 aa, Arachis hypogaea
LFEVKPDKKNPQLQDLDMMLTCVEIK

SEQ ID NO: 116 - protein, 38 aa, Arachis hypogaea
LFEVKPDKKNPQLQDLDMMLTCVEIKEGALMLPHFNSK

SEQ ID NO: 117 - protein, 27 aa, Arachis hypogaea
LIKNQKESHFVSARPQSQSQSPSSPEK

SEQ ID NO: 118 - protein, 9 aa, Arachis hypogaea
NNPFYFPSR

SEQ ID NO: 119 - protein, 10 aa, Arachis hypogaea
NNPFYFPSRR

SEQ ID NO: 120 - protein, 14 aa, Arachis hypogaea
NNPFYFPSRRFSTR

SEQ ID NO: 121 - protein, 21 aa, Arachis hypogaea
NNPFYFPSRRFSTRYGNQNGR

SEQ ID NO: 122 - protein, 17 aa, Arachis hypogaea
NPQLQDLDMMLTCVEIK

SEQ ID NO: 123 - protein, 29 aa, Arachis hypogaea
NPQLQDLDMMLTCVEIKEGALMLPHFNSK

SEQ ID NO: 124 - protein, 38 aa, Arachis hypogaea
NPQLQDLDMMLTCVEIKEGALMLPHFNSKAMVIVVVNK

SEQ ID NO: 125 - protein, 24 aa, Arachis hypogaea
NQKESHFVSARPQSQSQSPSSPEK

SEQ ID NO: 126 - protein, 40 aa, Arachis hypogaea
NQKESHFVSARPQSQSQSPSSPEKESPEKEDQEEENQGGK

SEQ ID NO: 127 - protein, 15 aa, Arachis hypogaea
NTLEAAFNAEFNEIR

SEQ ID NO: 128 - protein, 16 aa, Arachis hypogaea
NTLEAAFNAEFNEIRR

SEQ ID NO: 129 - protein, 30 aa, Arachis hypogaea
NTLEAAFNAEFNEIRRVLLEENAGGEQEER

SEQ ID NO: 130 - protein, 33 aa, Arachis hypogaea
NTLEAAFNAEFNEIRRVLLEENAGGEQEERGQR

SEQ ID NO: 131 - protein, 16 aa, Arachis hypogaea
QAKDLAFPGSGEQVEK

SEQ ID NO: 132 - protein, 9 aa, Arachis hypogaea
QFQNLQNHR

SEQ ID NO: 133 - protein, 50 aa, Arachis hypogaea
QFQNLQNHRIVQIEAKPNTLVLPKHADADNILVIQQGQATVTVANGNNRK

SEQ ID NO: 134 - protein, 7 aa, Arachis hypogaea
QKACESR

SEQ ID NO: 135 - protein, 16 aa, Arachis hypogaea
QKACESRCTKLEYDPR

SEQ ID NO: 136 - protein, 10 aa, Arachis hypogaea
QPGDYDDDRR

SEQ ID NO: 137 - protein, 13 aa, Arachis hypogaea
QPGDYDDDRRQPR

SEQ ID NO: 138 - protein, 14 aa, Arachis hypogaea
QPGDYDDDRRQPRR

SEQ ID NO: 139 - protein, 15 aa, Arachis hypogaea
QPREDWRRPSHQQPR

SEQ ID NO: 140 - protein, 16 aa, Arachis hypogaea
QPREDWRRPSHQQPRK

SEQ ID NO: 141 - protein, 9 aa, Arachis hypogaea
QPRREEGGR

SEQ ID NO: 142 - protein, 16 aa, Arachis hypogaea
QPRREEGGRWGPAGPR

SEQ ID NO: 143 - protein, 16 aa, Arachis hypogaea
REEEEDEDEEEEGSNR

SEQ ID NO: 144 - protein, 19 aa, Arachis hypogaea
REEEEDEDEEEEGSNREVR

SEQ ID NO: 145 - protein, 20 aa, Arachis hypogaea
REEEEDEDEEEEGSNREVRR

SEQ ID NO: 146 - protein, 6 aa, Arachis hypogaea
REEGGR

SEQ ID NO: 147 - protein, 13 aa, Arachis hypogaea
REEGGRWGPAGPR

SEQ ID NO: 148 - protein, 15 aa, Arachis hypogaea
REEGGRWGPAGPRER

SEQ ID NO: 149 - protein, 12 aa, Arachis hypogaea
RFSTRYGNQNGR

SEQ ID NO: 150 - protein, 14 aa, Arachis hypogaea
RFSTRYGNQNGRIR

SEQ ID NO: 151 - protein, 8 aa, Arachis hypogaea
RPSHQQPR

SEQ ID NO: 152 - protein, 15 aa, Arachis hypogaea
RVLLEENAGGEQEER

SEQ ID NO: 153 - protein, 18 aa, Arachis hypogaea
RVLLEENAGGEQEERGQR

SEQ ID NO: 154 - protein, 16 aa, Arachis hypogaea
RWSTRSSENNEGVIVK

SEQ ID NO: 155 - protein, 11 aa, Arachis hypogaea
SFNLDEGHALR

SEQ ID NO: 156 - protein, 23 aa, Arachis hypogaea
SFNLDEGHALRIPSGFISYILNR

SEQ ID NO: 157 - protein, 30 aa, Arachis hypogaea
SFNLDEGHALRIPSGFISYILNRHDNQNLR

SEQ ID NO: 158 - protein, 10 aa, Arachis hypogaea
SPPGERTRGR

SEQ ID NO: 159 - protein, 19 aa, Arachis hypogaea
SPPGERTRGRQPGDYDDDR

SEQ ID NO: 160 - protein, 11 aa, Arachis hypogaea
SRQFQNLQNHR

SEQ ID NO: 161 - protein, 51 aa, Arachis hypogaea
SRQFQNLQNHRIVQIEAKPNTLVLPKHADADNILVIQQGQATVTVANGNNR

SEQ ID NO: 162 - protein, 11 aa, Arachis hypogaea
SSENNEGVIVK

SEQ ID NO: 163 - protein, 14 aa, Arachis hypogaea
SSENNEGVIVKVSK

SEQ ID NO: 164 - protein, 22 aa, Arachis hypogaea
SSENNEGVIVKVSKEHVEELTK

SEQ ID NO: 165 - protein, 7 aa, Arachis hypogaea
SSPYQKK

SEQ ID NO: 166 - protein, 15 aa, Arachis hypogaea
SSPYQKKTENPCAQR

SEQ ID NO: 167 - protein, 28 aa, Arachis hypogaea
SSPYQKKTENPCAQRCLQSCQQEPDDLK

SEQ ID NO: 168 - protein, 20 aa, Arachis hypogaea
SVSKKGSEEEGDITNPINLR

SEQ ID NO: 169 - protein, 8 aa, Arachis hypogaea
TENPCAQR

SEQ ID NO: 170 - protein, 21 aa, Arachis hypogaea
TENPCAQRCLQSCQQEPDDLK

SEQ ID NO: 171 - protein, 23 aa, Arachis hypogaea
TENPCAQRCLQSCQQEPDDLKQK

SEQ ID NO: 172 - protein, 28 aa, Arachis hypogaea
TENPCAQRCLQSCQQEPDDLKQKACESR

SEQ ID NO: 173 - protein, 13 aa, Arachis hypogaea
TRGRQPGDYDDDR

SEQ ID NO: 174 - protein, 14 aa, Arachis hypogaea
TRGRQPGDYDDDRR

SEQ ID NO: 175 - protein, 23 aa, Arachis hypogaea
VAKISMPVNTPGQFEDFFPASSR

SEQ ID NO: 176 - protein, 49 aa, Arachis hypogaea
VAKISMPVNTPGQFEDFFPASSRDQSSYLQGFSRNTLEAAFNAEFNEIR

SEQ ID NO: 177 - protein, 14 aa, Arachis hypogaea
VLLEENAGGEQEER

SEQ ID NO: 178 - protein, 17 aa, Arachis hypogaea
VLLEENAGGEQEERGQR

SEQ ID NO: 179 - protein, 18 aa, Arachis hypogaea
VLLEENAGGEQEERGQRR

SEQ ID NO: 180 - protein, 22 aa, Arachis hypogaea
VLLEENAGGEQEERGQRRWSTR

SEQ ID NO: 181 - protein, 19 aa, Arachis hypogaea
VLQRFDQRSRQFQNLQNHR

SEQ ID NO: 182 - protein, 11 aa, Arachis hypogaea
VSKEHVEELTK

SEQ ID NO: 183 - protein, 22 aa, Arachis hypogaea
VSPLMLLLGILVLASVSATHAK

SEQ ID NO: 184 - protein, 7 aa, Arachis hypogaea
WGPAGPR

SEQ ID NO: 185 - protein, 9 aa, Arachis hypogaea
WGPAGPRER

SEQ ID NO: 186 - protein, 11 aa, Arachis hypogaea
WGPAGPRERER

SEQ ID NO: 187 - protein, 15 aa, Arachis hypogaea
WSTRSSENNEGVIVK

SEQ ID NO: 188 - protein, 26 aa, Arachis hypogaea
WSTRSSENNEGVIVKVSKEHVEELTK

SEQ ID NO: 189 - protein, 7 aa, Arachis hypogaea
YGNQNGR

SEQ ID NO: 190 - protein, 13 aa, Arachis hypogaea
YGNQNGRIRVLQR

SEQ ID NO: 191 - protein, 17 aa, Arachis hypogaea
YGNQNGRIRVLQRFDQR

SEQ ID NO: 192 - protein, 48 aa, Arachis hypogaea
YTARLKEGDVFIMPAAHPVAINASSELHLLGFGINAENNHRIFLAGDK

SEQ ID NO: 193 - protein, 18 aa, Arachis hypogaea
EQEWEEEEEDEEEEGSNR

SEQ ID NO: 194 - protein, 14 aa, Arachis hypogaea
GSEEEDITNPINLR

SEQ ID NO: 195 - protein, 14 aa, Arachis hypogaea
EGEQEWGTPGSEVR

SEQ ID NO: 196 - protein, 15 aa, Arachis hypogaea
IVQIEARPNTLVLPK

SEQ ID NO: 197 - protein, 10 aa, Arachis hypogaea
GAGSSQHQER

SEQ ID NO: 198 - protein, 7 aa, Arachis hypogaea
QQEQQFK

SEQ ID NO: 199 - protein, 17 aa, Arachis hypogaea
RPFYSNAPQEIFIQQGR

SEQ ID NO: 200 - protein, 12 aa, Arachis hypogaea
AHVQVVDSNGNR

SEQ ID NO: 201 - protein, 13 aa, Arachis hypogaea
FNLAGNHEQEFLR

SEQ ID NO: 202 - protein, 20 aa, Arachis hypogaea
NALFVPHYNTNAHSIIYALR

SEQ ID NO: 203 - protein, 13 aa, Arachis hypogaea
SPDIYNPQAGSLK

SEQ ID NO: 204 - protein, 13 aa, Arachis hypogaea
WLGLSAEYGNLYR

SEQ ID NO: 205 - protein, 23 aa, Arachis hypogaea
VYDEELQEGHVLVVPQNFAVAGK

SEQ ID NO: 206 - protein, 12 aa, Arachis hypogaea
SQSENFEYVAFK

SEQ ID NO: 207 - protein, 30 aa, Arachis hypogaea
AGQEQENEGGNIFSGFTPEFLAQAFQVDDR

SEQ ID NO: 208 - protein, 15 aa, Arachis hypogaea
GENESDEQGAIVTVR

SEQ ID NO: 209 - protein, 24 aa, Arachis hypogaea
QQYERPDEEEEYDEDEYEYDEEER

SEQ ID NO: 210 - protein, 12 aa, Arachis hypogaea
SQSDNFEYVAFK

SEQ ID NO: 211 - protein, 11 aa, Arachis hypogaea
TANDLNLLILR

SEQ ID NO: 212 - protein, 11 aa, Arachis hypogaea
TANELNLLILR

SEQ ID NO: 213 - protein, 33 aa, Arachis hypogaea
TDSRPSIANLAGENSFIDNLPEEVVANSYGLPR

SEQ ID NO: 214 - protein, 12 aa, Arachis hypogaea
AHVQVVDSNGDR

SEQ ID NO: 215 - protein, 12 aa, Arachis hypogaea
AQSENYEYLAFK

SEQ ID NO: 216 - protein, 45 aa, Arachis hypogaea
FNEGDLIAVPTGVAFWLYNDHDTDVVAVSLTDTNNNDNQLDQFPR

SEQ ID NO: 217 - protein, 20 aa, Arachis hypogaea
GADEEEEYDEDEYEYDEEDR

SEQ ID NO: 218 - protein, 15 aa, Arachis hypogaea
SSNPDIYNPQAGSLR

SEQ ID NO: 219 - protein, 24 aa, Arachis hypogaea
SVNELDLPILGWLGLSAQHGTIYR

SEQ ID NO: 220 - protein, 23 aa, Arachis hypogaea
VFDEELQEGHVLVVPQNFAVAGK

SEQ ID NO: 221 - protein, 23 aa, Arachis hypogaea
VYDEELQEGHVLVVPQNFAVAAK

SEQ ID NO: 222 - protein, 30 aa, Arachis hypogaea
AGQEEENEGGNIFSGFTPEFLAQAFQVDDR

SEQ ID NO: 223 - protein, 30 aa, Arachis hypogaea
AGQEEENEGGNIFSGFTPEFLEQAFQVDDR

SEQ ID NO: 224 - protein, 11 aa, Arachis hypogaea
FFVPPFQQSPR

SEQ ID NO: 225 - protein, 30 aa, Arachis hypogaea
TDSRPSIANLAGENSIIDNLPEEVVANSYR

SEQ ID NO: 226 - protein, 30 aa, Arachis hypogaea
AGQEEEDEGGNIFSGFTPEFLEQAFQVDDR

SEQ ID NO: 227 - protein, 30 aa, Arachis hypogaea
AGQEQENEGGNIFSGFTSEFLAQAFQVDDR

SEQ ID NO: 228 - protein, 15 aa, Arachis hypogaea
GENESEEEGAIVTVK

SEQ ID NO: 229 - protein, 15 aa, Arachis hypogaea
GENESEEQGAIVTVK

SEQ ID NO: 230 - protein, 18 aa, Arachis hypogaea
SPDEEEEYDEDEYAEEER

SEQ ID NO: 231 - protein, 12 aa, Arachis hypogaea
SQSEHFLYVAFK

SEQ ID NO: 232 - protein, 33 aa, Arachis hypogaea
TDSRPSIANLAGENSIIDNLPEEVVANSYGLPR

SEQ ID NO: 233 - protein, 33 aa, Arachis hypogaea
TDSRPSIANLAGENSVIDNLPEEVVANSYGLPR

SEQ ID NO: 234 - protein, 30 aa, Arachis hypogaea
TDSRPSIANQAGENSIIDNLPEEVVANSYR

SEQ ID NO: 235 - protein, 23 aa, Arachis hypogaea
VFDEELQEGQSLVVPQNFAVAAK

SEQ ID NO: 236 - protein, 14 aa, Arachis hypogaea
IMGEQEQYDSYDIR

SEQ ID NO: 237 - protein, 7 aa, Arachis hypogaea
QMVQQFK

SEQ ID NO: 238 - protein, 13 aa, synthetic construct
  REGION 1..13: note = Description of Artificial Sequence: Synthetic peptide
DLAFPGSGEQVEK

SEQ ID NO: 239 - protein, 11 aa, synthetic construct
  REGION 1..11: note = Description of Artificial Sequence: Synthetic peptide
GTGNLELVAVR

SEQ ID NO: 240 - protein, 10 aa, synthetic construct
  REGION 1..10: note = Description of Artificial Sequence: Synthetic peptide
GAGSSQHQER

SEQ ID NO: 241 - protein, 7 aa, synthetic construct
  REGION 1..7: note = Description of Artificial Sequence: Synthetic peptide
QQEQQFK

SEQ ID NO: 242 - protein, 17 aa, synthetic construct
  REGION 1..17: note = Description of Artificial Sequence: Synthetic peptide
RPFYSNAPQEIFIQQGR

SEQ ID NO: 243 - protein, 12 aa, synthetic construct
  REGION 1..12: note = Description of Artificial Sequence: Synthetic peptide
AHVQVVDSNGNR

SEQ ID NO: 244 - protein, 14 aa, synthetic construct
  REGION 1..14: note = Description of Artificial Sequence: Synthetic peptide
IMGEQEQYDSYDIR

SEQ ID NO: 245 - protein, 7 aa, synthetic construct
  REGION 1..7: note = Description of Artificial Sequence: Synthetic peptide
QMVQQFK

SEQ ID NO: 246 - protein, 13 aa, synthetic construct
  REGION 1..13: note = Description of Artificial Sequence: Synthetic peptide
DLAFPGSGEQVEK

SEQ ID NO: 247 - protein, 11 aa, synthetic construct
  REGION 1..11: note = Description of Artificial Sequence: Synthetic peptide
GTGNLELVAVR

SEQ ID NO: 248 - protein, 10 aa, synthetic construct
  REGION 1..10: note = Description of Artificial Sequence: Synthetic peptide
GAGSSQHQER

SEQ ID NO: 249 - protein, 7 aa, synthetic construct
  REGION 1..7: note = Description of Artificial Sequence: Synthetic peptide
QQEQQFK

SEQ ID NO: 250 - protein, 17 aa, synthetic construct
  REGION 1..17: note = Description of Artificial Sequence: Synthetic peptide
RPFYSNAPQEIFIQQGR

SEQ ID NO: 251 - protein, 12 aa, synthetic construct
  REGION 1..12: note = Description of Artificial Sequence: Synthetic peptide
AHVQVVDSNGNR

SEQ ID NO: 252 - protein, 14 aa, synthetic construct
  REGION 1..14: note = Description of Artificial Sequence: Synthetic peptide
IMGEQEQYDSYDIR

SEQ ID NO: 253 - protein, 7 aa, synthetic construct
  REGION 1..7: note = Description of Artificial Sequence: Synthetic peptide
QMVQQFK